(19) United States
(12) Patent Application Publication
(10) Pub. No.: US 2025/0186438 A1
(43) Pub. Date: Jun. 12, 2025

(54) TREATMENT OF CLEAR CELL RENAL CELL CARCINOMA

(71) Applicant: RACE ONCOLOGY LTD, Sydney, New South Wales (AU)

(72) Inventors: Daniel TILLETT, Woollahra, New South Wales (AU); Nicole Margaret VERRILLS, Merewether, New South Wales (AU); Heather Constance MURRAY, Belmont North, New South Wales (AU); Joshua Stephen Sidney BRZOZOWSKI, Fennell Bay, New South Wales (AU)

(21) Appl. No.: 18/844,586

(22) PCT Filed: Mar. 9, 2023
(86) PCT No.: PCT/AU2023/050163

§ 371 Date: Sep. 6, 2024

(30) Foreign Application Priority Data
Mar. 9, 2022 (AU) 2022900578

(51) Int. Cl. A61K 31/506; A61K 31/4178; A61K 31/47; A61K 45/06; A61P 35/00

(52) U.S. Cl. CPC A61K 31/506; A61K 31/4178; A61K 31/47; A61K 45/06; A61P 35/00

(57) ABSTRACT

The present invention provides methods for treating patients with ccRCC and other VHL(−) cancers, the method comprising administering to the patient a therapeutically effective amount of a protein kinase inhibitor and a therapeutically effective amount of at least a second agent comprising bisantrene or a derivative thereof, or a pharmaceutically acceptable salt of bisantrene or derivative thereof. Also provided by the present invention are pharmaceutical compositions and kits for the treatment of ccRCC and other VHL(−) cancers as well as the use of such compositions for the manufacture of medicaments for the treatment of ccRCC and other VHL(−) cancers.

### FIELD OF THE INVENTION

This invention is directed to the use of bisantrene in combination with protein kinase inhibitors to treat clear cell renal cell carcinoma (ccRCC) and other cancers with inactive von Hippel-Lindau (VHL) tumour suppressor genes. The present invention also contemplates pharmaceutical compositions and kits for such treatments, as well as the use of such compositions for the manufacture of medicaments for such treatments.

### BACKGROUND OF THE INVENTION

Kidney cancer is one of the top 10 most common cancers. Renal cell carcinomas (RCC) account for 3.8% of all new cancers. Approximately 80-85% of renal cell carcinomas (RCC) are clear cell renal cell carcinomas (ccRCC). The other 15% to 20% are non-clear cell RCC which comprise a diverse group of histologic subtypes, each with varying molecular profiles. According to the American Cancer Society, the lifetime risk for developing kidney cancer in men is 1 in 47 and in women is 1 in 82.

Smoking and obesity are established risk factors for RCC development. Several hereditary types of RCC also exist, with von Hippel-Lindau (VHL) disease being the most common. VHL disease is caused by an autosomal-dominant mutation in the VHL gene which predisposes individuals to ccRCC and a range of other cancers. VHL loss-of-function has also been shown to be important in sporadic ccRCC. A large number of studies have reported VHL loss-of-function changes VHL(−) in more than 90% of sporadic ccRCCs caused by allele loss, mutation, and promoter methylation.

In the United States, there were approximately 65,000 new cases of kidney cancer with 15,000 deaths in 2019. Patients with advanced disease are often asymptomatic with around 20% of patients having metastatic disease at the time of diagnosis. The most important prognostic determinants of 5-year survival are the tumor stage, grade, local extent of the tumor, presence of regional nodal metastases, and evidence of metastatic disease at presentation. RCC primarily metastasizes to the lung, lymph nodes, bone, liver, adrenal gland, and brain. The 5-year survival rate for metastatic ccRCC is as low as 12%.

Surgery is the first line of treatment for Stage I to III ccRCC with cytoreductive nephrectomy followed by systemic therapy typically used to treat metastatic disease. RCC is not highly responsive to cytotoxic chemotherapy or radiotherapy. Targeted therapies have benefitted a growing number of RCC patients. Targeted agents such as vascular endothelial growth factor (VEGF) binding monoclonal antibodies (bevacizumab), oral tyrosine kinase inhibitors with potent activity against VEGF receptors (for example, axitinib, cabozantinib, lenvatinib, pazopanib, sorafenib, sunitinib and tivozanib), and mammalian target of rapamycin inhibitors (everolimus and temsirolimus), have provided new treatment alternatives in recent years.

Most ccRCC tumor cells express PD-L1 on the cell membrane which helps them evade immune attack. The immune checkpoint inhibitors, PD-1 inhibition or PD-L1 inhibition block this pathway, releasing the “off switch” on the immune system, increasing the ability of cytotoxic T cells to kill tumor cells. CTLA-4 inhibition stops autoreactive T cells during the immune priming phase, thereby supporting the activation and proliferation of effector T cells. The FDA has approved two PD-1 inhibitors (nivolumab and pembrolizumab), one PD-L1 inhibitor (avelumab), and one CTLA-4 inhibitor (ipilimumab) for use in ccRCC. Other PD-1/PD-L1 immune checkpoint inhibitors of potential consideration include atezolizumab, bevacizumab, cemiplimab, dostarlimab, and durvalumab.

Despite these new treatment options, many patients have disease progression due to the development of drug resistance or bypass pathways. Therefore, there is a need for new drugs, new combinations of drugs or new combined drug treatments for the treatment of ccRCC and other VHL(−) cancers.

### SUMMARY OF THE INVENTION

The present invention provides a new paradigm for treating ccRCC and other VHL(−) cancers by the administration of bisantrene in combination with a protein kinase inhibitor. This meets the needs for new treatments that improve the clinical outcomes of patients with ccRCC and other VHL(−) cancers, especially patients with protein kinase inhibitor resistant ccRCC.

Bisantrene is an antineoplastic agent that has multiple mechanisms of action, including DNA intercalation, inhibition of topoisomerase and the fat mass and obesity-associated protein (FTO), and activation of the immune system.

Surprisingly, it has been found through the course of these studies that bisantrene and pharmaceutically acceptable salts thereof act synergistically with protein kinase inhibitors against clear cell renal carcinoma cancer cells.

One aspect of the invention therefore provides a method of treating a patient with ccRCC or other VHL(−) cancer, said method comprising administering to said patient a therapeutically effective amount of a protein kinase inhibitor, which may be a tyrosine kinase inhibitor, and a therapeutically effective amount of at least a second agent comprising bisantrene or a derivative thereof, or a pharmaceutically acceptable salt of bisantrene or derivative thereof.

According to certain embodiments the protein kinase inhibitor inhibits one or more of VEGFR1, VEGFR2, VEGFR3, DDR1, DDR2, RET and RIPK2. According to certain embodiments the protein kinase inhibitor inhibits mTOR.

According to certain embodiments, the at least one protein kinase inhibitor is selected from the group consisting of:
(a) pazopanib;
(b) lenvatinib;
(c) cabozantinib;
(d) everolimus;
(e) sorafenib;
(f) sunitinib;
(g) temsirolimus;
(h) mitomycin C;
(i) axitinib;
(j) tivozanib; and
(k) belzutifan.

According to certain embodiments, the at least one protein kinase inhibitor is selected from the group consisting of:
(a) pazopanib;
(b) lenvatinib; and
(c) cabozantinib.

According to certain embodiments, the second agent is bisantrene or a pharmaceutically acceptable salt thereof.

According to certain embodiments, the method comprises administering to a patient a therapeutically effective amount of one protein kinase inhibitor and a therapeutically effective amount of bisantrene or a pharmaceutically acceptable salt thereof.

According to certain embodiments, the method comprises treatment of protein kinase inhibitor resistant ccRCC.

The method can further comprise the step of determining the VHL mutation and epigenetic status of a patient's cancer. The VHL mutational or epigenetic status can be determined prior to initiation of treatment or, alternatively, during treatment as a marker of the progress of treatment. The VHL mutation or epigenetic status of the cancer can be determined by next-generation sequencing, by RT-PCR, by ELISA assay with a suitable antibody directed against VHL, by RNA hybridization assays, or any other method known to the art.

According to certain embodiments, the method may further comprise administration of at least one additional therapeutic agent, optionally a checkpoint inhibitor drug or an immunomodulator for the treatment of ccRCC or other VHL(−) cancer. The at least one additional therapeutic agent is selected from the group consisting of: atezolizumab; avelumab; bevacizumab; cemiplimab; dostarlimab; durvalumab; interleukin-2; ipilimumab; nivolumab; pembrolizumab; and proleukin.

According to certain embodiments, the method may comprise administering said at least one protein kinase inhibitor to said patient prior to, simultaneously with, or after administration of said second agent to said patient.

According to other embodiments, the method may comprise administering said at least one protein kinase inhibitor and the second agent to said patient at the same time, optionally in a single composition.

According to certain embodiments, the treatment has synergistic results against ccRCC and other VHL(−) cancers compared to a method wherein said at least one protein kinase or said second agent is administered alone. In certain embodiments, the dose of said at least one protein kinase inhibitor is at least 20% lower than the dose required of said at least one protein kinase inhibitor agent when administered without said second agent to achieve the same targeted outcome.

Another aspect of the invention provides a pharmaceutical composition for the treatment of ccRCC or other VHL(−) cancer by embodiments of treatment methods as described above, said compositions comprising at least one protein kinase inhibitor, which may be a tyrosine kinase inhibitor, as described above, and a second agent as described above comprising bisantrene or a derivative thereof, or a pharmaceutically acceptable salt of bisantrene or derivative thereof. Said compositions may comprise further active agents as described above.

Another aspect of the invention provides the use of a composition according to the invention, as described above, for the manufacture of a medicament for the treatment of ccRCC or other VHL(−) cancer, optionally protein kinase inhibitor resistant ccRCC in a patient.

Yet another aspect of the invention provides a kit for the treatment of ccRCC or other VHL(−) cancer, optionally protein kinase inhibitor resistant ccRCC by embodiments of methods of the present invention, as described above, said kit comprising at least one protein kinase inhibitor, optionally a tyrosine kinase inhibitor, as described above, and at least a second agent as described above, said second agent comprising bisantrene or a derivative thereof, or a pharmaceutically acceptable salt of bisantrene or derivative thereof. Kits according to the present invention may comprise instructions for administering said at least one protein kinase inhibitor to said patient prior to, simultaneously with, or after administering said second agent to said patient. Alternatively, kits according to the present invention may comprise instructions for administering said at least one protein kinase inhibitor and the second agent to said patient at the same time.

### BRIEF DESCRIPTION OF THE DRAWINGS

The following invention will become better understood with reference to the specification, appended claims, and accompanying drawings, where the terms “Zan” or “Zantrene” are alternative terms for bisantrene.

FIG. 1 shows a Webb analysis of bisantrene-everolimus or -sunitinib drug combinations in 786-O cells. A) Cell viability in response to different dose ranges of Everolimus and Sunitinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 2 shows a Webb analysis of bisantrene-sorafenib or -pazopanib drug combinations in 786-O cells. A) Cell viability in response to different dose ranges of Sorafenib and Pazopanib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (red); between −0.1 to 0.1 is additive (green); and >0.1 is antagonistic (yellow).

FIG. 3 shows a Webb analysis of bisantrene-lenvatinib or -cabozantinib drug combinations in 786-O cells. A) Cell viability in response to different dose ranges of Lenvatinib and Cabozantinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 4 shows a Webb analysis of bisantrene-everolimus or -sunitinib drug combinations in Caki-1 cells. Cell viability in response to different dose ranges of Everolimus and Sunitinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 5 shows a Webb analysis of bisantrene-sorafenib, or -pazopanib drug combinations in Caki-1 cells. A) Cell viability in response to different dose ranges of Sorafenib and Pazopanib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 6 shows a Webb analysis of bisantrene-lenvatinib or -cabozantinib drug combinations in Caki-1 cells. A) Cell viability in response to different dose ranges of Lenvatinib and Cabozantinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 7 shows a Webb analysis of bisantrene-everolimus or -sunitinib drug combinations in Caki-2 cells. Cell viability in response to different dose ranges of Everolimus and Sunitinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 8 shows a Webb analysis of bisantrene-sorafenib, or -pazopanib drug combinations in Caki-2 cells. A) Cell viability in response to different dose ranges of Sorafenib and Pazopanib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 9 shows a Webb analysis of bisantrene-lenvatinib or -cabozantinib drug combinations in Caki-2 cells. A) Cell viability in response to different dose ranges of Lenvatinib and Cabozantinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 10 shows a Webb analysis of bisantrene-everolimus or -sunitinib drug combinations in RCC4 EV cells. Cell viability in response to different dose ranges of Everolimus and Sunitinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 11 shows a Webb analysis of bisantrene-sorafenib, or -pazopanib drug combinations in RCC4 EV cells. A) Cell viability in response to different dose ranges of Sorafenib and Pazopanib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 12 shows a Webb analysis of bisantrene-lenvatinib or -cabozantinib drug combinations in RCC4 EV cells. A) Cell viability in response to different dose ranges of Lenvatinib and Cabozantinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 13 shows a Webb analysis of bisantrene-everolimus or -sunitinib drug combinations in RCC4 VHL cells. Cell viability in response to different dose ranges of Everolimus and Sunitinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 14 shows a Webb analysis of bisantrene-sorafenib or -pazopanib drug combinations in RCC4 VHL cells. A) Cell viability in response to different dose ranges of Sorafenib and Pazopanib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 15 shows a Webb analysis of bisantrene-lenvatinib or -cabozantinib drug combinations in RCC4 VHL cells. A) Cell viability in response to different dose ranges of Lenvatinib and Cabozantinib in combination with Zantrene, as indicated. Experimental data is shown for each drug alone and the combinations. The ‘Expected value’ is calculated using the method of Webb and shows the expected value if the drug combination was additive. Therefore any experimental values below this line are considered synergistic. At or near the line is additive; and above the line is antagonistic. B) Webb analysis for all drug combination doses tested where a result of <−0.1 indicates synergy (*); between −0.1 to 0.1 is additive (&); and >0.1 is antagonistic (#).

FIG. 16 shows bisantrene plus a targeted or chemotherapeutic agent to overcome treatment resistance of ccRCC and other VHL(−) cancers.

FIG. 17 shows bisantrene plus a checkpoint inhibitor combination trial design to overcome immunotherapy resistance of ccRCC and other VHL(−) cancers.

FIG. 18 shows the association between bisantrene sensitivity and VHL status of isogenic renal cancer cell lines RCC4 EV (VHL(−) mutant) and RCC4 VHL (VHL rescue line). (A) IC₅₀ values based on VHL status, compared using t-test. (B) Direct comparison of Zantrene sensitivity in the RCC4 isogenic cell line pair. Mean+/−SEM, n=3.

FIG. 19 shows the association between bisantrene sensitivity and VHL status as assessed using clonogenicity assays. (A) IC₅₀ values were compared between VHL mutant and wildtype cell lines. NS, not significant, unpaired t-test. (B) Colonies observed for the RCC4 EV and RCC4 VHL rescue cell lines. n=4, *p<0.05, paired t-test.

### DEFINITIONS

As used herein, “treating” means affecting a subject/patient, tissue or cell to obtain a desired pharmacological and/or physiological effect and includes inhibiting a condition, i.e. slowing or arresting its development; or relieving or ameliorating the effects of the condition i.e., cause reversal or regression of the effects of the condition.

As used herein, “preventing” means preventing a condition from occurring in a cell, tissue or subject that may be at risk of having the condition, but does not necessarily mean that condition will not eventually develop, or that a subject will not eventually develop a condition. Preventing includes delaying the onset of a condition in a cell, tissue or subject.

As used herein, the term “subject” or “patient” refers to a mammal. The mammal may be a human or a non-human. Examples of non-humans include primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Typically, the mammal is a human or a non-human primate. More typically, the mammal is a human.

The term “composition” encompasses compositions and formulations comprising the active pharmaceutical ingredient (for example, “protein kinase inhibitor” and “second agent comprising bisantrene or a derivative of bisantrene, or a pharmaceutically acceptable salt of bisantrene or cardioprotective derivative of bisantrene”) with excipients or carriers, and also compositions and formulations with a carrier. In pharmaceutical compositions, the excipient or carrier is “pharmaceutically acceptable” meaning that it is not biologically or otherwise undesirable, i.e., the material may be incorporated into a pharmaceutical composition administered to a patient without causing any undesirable biological effects or interacting in a deleterious manner with any of the other components of the composition in which it is contained. Supplementary active ingredients can also be incorporated into the compositions.

By “pharmaceutically acceptable” such as in the recitation of a “pharmaceutically acceptable salt” or a “pharmaceutically acceptable excipient or carrier” is meant herein a material that is not biologically or otherwise undesirable, i.e., the material may be incorporated into a pharmaceutical composition administered to a patient without causing any undesirable biological effects or interacting in a deleterious manner with any of the other components of the composition in which it is contained.

The term “effective amount” or “therapeutically effective amount” refers to the quantity of an active pharmaceutical ingredient that is sufficient to yield a desired therapeutic response. The specific effective amount or therapeutically effective amount will vary with such factors as the particular condition being treated, the age, body weight, general health, physical condition, gender and diet of the subject, the duration of the treatment, the nature of concurrent therapy (if any), and the severity of the particular condition.

As used herein, “carrier” includes any and all solvents, dispersion media, vehicles, coatings, diluents, bulking agents, carrier solutions, suspensions, colloids, and forming and binding agents, any or all of which may include other pharmaceutical excipients as known in the art, including lubricants, antibacterial and antifungal agents, isotonic and absorption delaying agents, buffers, antioxidants, other stabilisers, including physical stabilisers such as thickeners and viscosity enhancers, colouring agents, flavouring and sweetening agents, and the like. The use of such media and agents for pharmaceutical active substances is well known in the art. Except insofar as any conventional media or agent is incompatible with the active ingredient, its use in the therapeutic compositions is contemplated.

As used herein, “administration” or “administer” or “administering” refers to dispensing, applying, or tendering one or more agents to a subject. Administration can be performed using any of a number of methods known in the art. For example, “administering” as used herein is meant via infusion (intravenous administration (i.v.)), parenteral administration. By “parenteral” is meant intravenous, subcutaneous and intramuscular administration.

### DETAILED DESCRIPTION OF THE INVENTION

The present disclosure relates to a method for the treatment of ccRCC or other VHL(−) cancer, especially protein kinase inhibitor resistant ccRCC, comprising administering a therapeutically active amount of bisantrene in combination with a protein kinase inhibitor, especially a tyrosine kinase inhibitor (“TKI”).

### Protein Kinase Inhibitors

Protein kinases are protein enzymes that phosphorylate amino acid sidechains that bear hydroxyl groups on target proteins, namely serine, threonine, and tyrosine, as well as other targets, typically in signaling pathways. In fact, many signaling pathways comprise cascades of protein kinases. This phosphorylation in many cases causes conformational changes in the target protein which may in turn result in activation or inhibition of the target protein's activity or modify its physical properties (thereby modulating its interactions). Thus protein phosphorylation and dephosphorylation events may therefore activate or inhibit particular physiological pathways. Dysregulation of such signaling, such as through mutations that cause, for example, constitutive expression or unregulated activity of particular proteins or pathways may be involved in cancers and/or allow for their survival. Dysregulation of expression and/or activity of various protein kinases, such as EGFR, VEGFR, HER, ALK, RAS and RAF (to name a few) has been associated with increased growth, proliferation, motility and survival of various tumour cells, including ccRCC cells.

There is a very wide range of protein kinases known in the art. In fact, the human genome is known to include 518 protein kinase genes. A very large number of protein kinases have been known to be associated with cancers, including ccRCC, which is not surprising given that they are associated with signaling pathways and activation or inhibition of pathways associated with growth, especially angiogenesis, as well as transmembrane signaling.

Protein kinases associated with cancers, especially kidney cancers such as ccRCC include, for example, tyrosine kinases, serine/threonine kinases, cyclin-dependent kinases, aurora kinases, mTOR, and mitogen-activated protein kinases amongst others, and these may include, for example, kinases such as AATK, ABL1, AKT2, ALK, AURKA, AXL, CHK2, DDR1, DDR2, EML4-ALK, EPHA2, EPHA5, ERBB2, EGFR, FIP1L1-PDGFRA, FTL3, HER, HIF, KIT, MET, p21Cip1, p27Kip1, PDGFR, PKD1, RET, retinoblastoma protein (RB), RIPK2, SCH1, VEGFR1, VEGFR2, and VEGFR3.

It is important to note that kinases as mentioned above may have a primary target, but may also phosphorylate other targets/moieties. For example, tyrosine kinases may also phosphorylate serine and/or threonine hydroxyl moieties. Similarly, a serine/threonine kinase may also phosphorylate a tyrosine hydroxyl moiety. Similarly, although a kinase may primarily target one protein, it may also phosphorylate other proteins. Thus, a tyrosine kinase may predominantly target, or be known to target a tyrosine hydroxyl on particular proteins (often receptors), but may also be able to phosphorylate hydroxyl moieties on serine and/or threonine, and/or on other proteins. This variability in specificity of target, as known in the art, is incorporated herein within the context of protein kinases.

A wide range of protein kinase inhibitors have been developed for the treatment of various cancers, including imatinib, being the first rationally designed protein kinase inhibitor to achieve FDA approval in 2001. Since then, protein kinase inhibitors such as gefitinib, erlotinib, dasatinib, nilotinib, lapatinib, osimertinib, olmutinib, lorlatinib, capmatinib, tepotinib, selpercatinib, pralsetinib, selumetinib, trametinib, afatinib, ibrutinib, dabrafenib, ponatinib, bosutinib, radotinib, idelalisib, crizotinib, vemurafenib, xolitinib, olmutinib, Mitomycin C, temsirolimus, everolimus, axitinib, sorafinib, sunitinib, cabozantinib, lenvatinib, pazopanib and tivozanib to name but a few.

Protein kinase inhibitors currently approved for treatment of ccRCC include, for example, temsirolimus, axitinib, belzutifan, cabozantinib, everolimus, lenvatinib, mitomycin C, pazopanib, sorafenib, sunitinib and tivozanib.

Axitinib, cabozantinib, pazopanib, sorafenib, sunitinib are small molecule tyrosine kinase inhibitors, including targets such as the VEGFR family. Bezultifan is an inhibitor of hypoxia inducible factor (HIF)-2alpha (HIF-2a). Everolimus and temsirolimus are inhibitors of the mammalian target of rapamycin (mTOR) complex. Lenvatinib is a multiple kinase inhibitor that acts on VEGFR1, 2 and 3, as well as fibroblast growth factor receptors (FGFR) 1, 2, 3 and 4, platelet-derived growth factor receptor (PDGFR) alpha, c-Kit, and the RET proto-oncogene. Mitomycin C is a potent DNA crosslinking chemotherapeutic. Tivozanib is a multiple kinase inhibitor that acts on VEGFR1, 2 and 3.

In addition to the VEGFR family of protein kinases, common targets for lenvatinib, cabozantinib and pazopanib include DDR1 and DDR2 (discoidin domain receptors 1 and 2), RET (“REarranged during Transfection”) and RIPK2 (Receptor interacting serine/threonine kinase 2). RET has also been identified as a target of Sunitinib and Axitinib, and AXL and MET as additional cabozantinib targets.

Notwithstanding the success in use of protein kinase inhibitors to treat various cancers, including ccRCC and other VHL(−) cancers, many patients have disease progression due to the development of drug resistance or bypass pathways.

The present studies have shown surprising synergies between bisantrene and protein kinase inhibitors used in treatment of ccRCC and other VHL(−) cancers. Such synergy is expected to assist in at least reversing or mitigating if not abrogating development of resistance, and is also expected to have the added benefit of allowing lower dosing of protein kinase inhibitors and/or bisantrene, and thereby reducing any adverse side-effects.

### Bisantrene and Derivatives or Analogs Thereof, and Pharmaceutically Acceptable Salts

Bisantrene has immunologic properties that might be responsible for some of its activities, and which may make this agent a useful tool in the combinatorial supercoiling and initiates strand breaks in association with DNA associated proteins. This results from the inhibition of the enzyme topoisomerase II, which relaxes DNA coiling during replication. It was found that while inactive orally, intravenously (i.v.), intraperitoneally (i.p.), or subcutaneously (s.c.), the drug was effective in cancer models using colon 26, Lewis lung, Ridgway osteosarcoma, B16, Lieberman plasma cell, P388 or L1210 cancer cells. Activity in clonogenic assays from 684 patients was seen in breast, small cell lung, large cell lung, squamous cell lung, ovarian, pancreatic, renal, adrenal, head and neck, sarcoma, gastric, lymphoma and melanoma tumor cells, but not in colorectal cancer. Importantly, a lack of cross resistance with adriamycin and mitoxantrone was found. Subsequent to treatment with bisantrene, and for 4 weeks thereafter, macrophages could be isolated from peritoneal exudate that had cytostatic anti-proliferative activity in cultures of P815 (mastocytoma) tumor cells. Moreover, the supernatants from bisantrene activated macrophages also had a protective cytostatic effect in the tumor cell cultures. Further work revealed that macrophages activated with bisantrene and adoptively transferred to mice with EL-4 lymphomas more than doubled their median survival time, with 7 of 10 mice in the group being cured. Multiple administrations of activated macrophages were more effective than a single administration. Bisantrene is an unusual agent with direct cytotoxic action as well as genomic and immunologic methods of action including as a potent inhibitor (IC₅₀ 142 nM) of the fat mass and obesity-associated protein (FTO), an RNA N6-methyladenosine (m6A) demethylase (Su, R., Dong, L., Li, Y., Gao, M., Han, L., Wunderlich, M., et al. (2020), “Targeting FTO Suppresses Cancer Stem Cell Maintenance and Immune Evasion”, Cancer Cell, 38(1): 79-96.e11).

The chemical name for bisantrene is 9, 10-anthracenedicarboxaldehyde-bis [(4, 5-dihydro-1H-imidazole-2-yl) hydrazine]dihydrochloride, and it was originally classed as an anthracycline chemotherapeutic agent. As used herein, the term “bisantrene” refers to bisantrene or any pharmacologically compatible salt form, not only bisantrene dihydrochloride, unless the dihydrochloride or another specific pharmacologically compatible salt form is specifically indicated. Typically, the pharmacologically compatible salt of bisantrene is bisantrene dihydrochloride for most pharmacological applications. These are drugs with planar structures based around a resonant aromatic ring structure that intercalates within the helices of DNA and disrupt various functions, including replication, presumably due to a strong inhibitory effect on the enzyme topoisomerase II. It was found that, like other anthracyclines, it could kill tumor cells in clonogenic assays and intercalate with DNA, where it inhibits both DNA and RNA synthesis. The primary chemotherapeutic mechanism for bisantrene is its preferential binding to A-T rich regions where it effects changes to

Recent studies have identified that bisantrene suppresses immune checkpoint gene expression and immune evasion via enzymatic inhibition of the FTO RNA demethylase as described in R. Su. et al. (2020), “Targeting FTO Suppresses Cancer Stem Cell Maintenance and Immune Evasion”, Cancer Cell, 38(1): 79-96.e11.

Bisantrene has also been found to have non-immunologic telomeric effects. Bisantrene binds to DNA at a site called a G-quadruplex, in which 4 guanines are associated by folding. Stabilization of the G-quadruplex can interfere with telomere-telomerase interaction and thus inhibit the activity of telomerase in various ways, including the displacement of telomerase binding proteins. Since the level of topoisomerase II inhibition does not always correlate with cytotoxic efficacy, alternative mechanisms may play a role in the actions of bisantrene. Analogs of bisantrene have been made in an attempt to improve upon the anti-telomerase activity; these analogs are described further below. Human melanoma (SK-Mel5) and colon cancer (LoVo) tumor cells were observed to lose their proliferative ability in the presence of these agents. Apoptosis was not observed; however a loss of immortality was seen, with treated cells reacquiring the ability to become senescent, age, and die.

As detailed above, in addition to direct antineoplastic effects related to the activity of bisantrene as a DNA intercalator, bisantrene also possesses other mechanisms of action, including immunopotentiation. These mechanisms are described in: (i) N. R. West et al. (2011), “Tumor-Infiltrating Lymphocytes Predict Response to Anthracycline-Based Chemotherapy in Estrogen-Resistant Breast Cancer”, Breast Canc. Res. 13: R126, which concludes that the level of tumor-infiltrating lymphocytes is correlated with a response to the administration of anthracycline-based agents; the markers associated with tumor-infiltrating lymphocytes (TIL) include CD19, CD3D, CD48, GZMB, LCK, MS4A1, PRF1, and SELL; (ii) L. Zitvogel et al. (2008), “Immunological Aspects of Cancer Chemotherapy”, Nature Rev. Immunol. 8: 59-73, which states that DNA damage, such as that produced by intercalating agents such as bisantrene, induces the expression of NKG2D ligands on tumor cells in an ATM-dependent and CHK1-dependent (but p53-independent) manner; NKG2D is an activating receptor that is involved in tumor immunosurveillance by NK cells, NK T cells, γδ T cells and resting (in mice) and/or activated (in humans) CD8+ T cells, and also states that anthracycline-based agents may act as immunostimulators, particularly in combination with IL-12; such agents also promote HMGB1 release and activate T cells; (iii) D. V. Krysko et al. (2011), “TLR2 and TLR9 Are Sensors of Apoptosis in a Mouse Model of Doxorubicin-Induced Acute Inflammation”, Cell Death Different. 18: 1316-1325, which states that anthracycline-based antibiotics induce an immunogenic form of apoptosis that has immunostimulatory properties mediated by MyD88, TLR2, and TLR9; (iv) C. Ferraro et al. (2000), “Anthracyclines Trigger Apoptosis of Both G0-G1 and Cycling Peripheral Blood Lymphocytes and Induce Massive Deletion of Mature T and B Cells”, Cancer Res. 60: 1901-1907, which stated that anthracyclines induce apoptosis and ceramide production, as well as activate caspase-3 in resting and cycling cells; the apoptosis induced is independent from CD95-L/CD95 and TNF/TNF-R; (v) K. Lee et al. (2009), “Anthracycline Chemotherapy Inhibits HIF-1 Transcriptional Activity and Tumor-Induced Mobilization of Circulating Angiogenic Cells”, Proc. Natl. Acad. Sci. USA 106: 2353-2358, which provides another antineoplastic mechanism for anthracycline-based antibiotics, namely inhibition of HIF-1 mediated gene transcription, which, in turn, inhibits transcription of VEGF required for angiogenesis; HIF-1 also activates transcription of genes encoding glucose transporter GLUT1 and hexokinases HK1 and HK2, which are required for the high level of glucose uptake and phosphorylation that is observed in metastatic cancer cells, and pyruvate dehydrogenase kinase 1 (PDK1), which shunts pyruvate away from the mitochondria, thereby increasing lactate production; patients with HIF-1a overexpression based on immunohistochemical results were suggested to be good candidates for treatment with anthracycline-based antibiotics.

Several clinical trials have investigated the pharmacokinetics of bisantrene in humans. In one trial of patients given a 90 min infusion at 260 mg/m2 a biphasic elimination with an initial half-life of 65±15 min, a terminal half-life of 1142±226 min, and a steady state volume of distribution (Vdss) of 1845 L/m2. Plasma clearance in this trial was 735 mL/min/m2, with 11.3% of the administered dose excreted unchanged in the urine in 24 h. In another trial, doses of 80-250 mg/m2 were assessed, and the initial and terminal half-lives were 0.6 h and 24.7 h. respectively, with a clearance of 1045.5±51.0 mL/kg/h and a calculated volume of distribution of 42.1±5.9 L/kg. In this study only 3.4±1.1% of the administered dose was found in the urine over 96 h. In three other single dose studies triphasic elimination was reported, one with t½ α, β, and γ of 3.44 min 1.33 h and 26.13 h, respectively, another was 3 min, 1 h, and 8 h respectively, and the last revealed clearances of 0.1 h, 1.9 h and 43.9 h, respectively. In one report a large volume of distribution (687 L/m2) was interpreted as tissue sequestration of the drug with a subsequent depot effect. In a 72 h infusion study, a plasma concentration of 12±6 ng/mL was observed at a dose of 56 mg/m2, while a dose of 260 mg/m2 resulted in a plasma concentration of 55±8 ng/mL In this trial plasma clearance was 1306±179 mL/min/m2 with urinary excretion of 4.6% of the dose in 24 h. Finally, in another study, a 5-day schedule of 60 min infusions revealed a t½ α and β of 0.9 and 9.7 h, respectively with 7.1% of the dose excreted in the urine.

The structure of bisantrene dihydrochloride is shown in Formula (I)

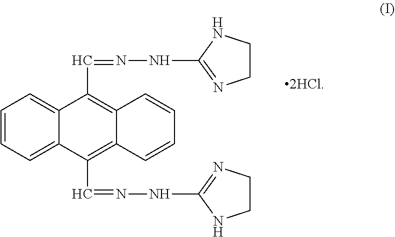 [SMILES: C(=NNC1=NCCN1)c1c2ccccc2c(C=NNC2=NCCN2)c2ccccc12]

Bisantrene dihydrochloride is a tricyclic aromatic compound with the chemical name, 9,10-anthracenedicarboxaldehyde bis[(4,5 dihydro-1H-imidazo-2-yl)hydrazine]dihydrochloride. The molecular formula of bisantrene dihydrochloride is C₂₂H₂₂N₈·2HCl and the molecular weight, 471.4. The alkylimidazole side chains are very basic and, at physiologic pH, are positively charged. This is believed to facilitate electrostatic attractions to negatively charged ribose phosphate groups in DNA.

Bisantrene is typically administered intravenously, either centrally or peripherally.

Various formulations suitable for use in the administration of bisantrene or derivatives or analogs thereof are known in the art. U.S. Pat. No. 4,784,845 to Desai et al. discloses a composition of matter for delivery of a hydrophobic drug (i.e., bisantrene or a derivative or analog thereof) comprising: (i) the hydrophobic drug; (ii) an oleaginous vehicle or oil phase that is substantially free of butylated hydroxyanisole (BHA) or butylated hydroxytoluene (BHT); (iii) a co-surfactant or emulsifier; (iv) a co-surfactant or auxiliary emulsifier; and (v) benzyl alcohol as a co-solvent. U.S. Pat. No. 4,816,247 by Desai et al. discloses a composition of matter for delivery by intravenous, intramuscular, or intraarticular routes of hydrophobic drugs (such as bisantrene or a derivative or analog thereof) comprising: (i) the hydrophobic drug; (ii) a pharmaceutically acceptable oleaginous vehicle or oil selected from the group consisting of: (a) naturally occurring vegetable oils and (b) semisynthetic mono-, di-, and triglycerides, wherein the oleaginous vehicle or oil is free of BHT or BHA; (iii) a surfactant or emulsifier; (iv) a co-surfactant or emulsifier; (v) an ion-pair former selected from C₆-C₂₀ saturated or unsaturated aliphatic acids when the hydrophobic drug is basic and a pharmaceutically acceptable aromatic amine when the hydrophobic drug is acidic; and (vi) water. U.S. Pat. No. 5,000,886 to Lawter et al. and U.S. Pat. No. 5,143,661 to Lawter et al. disclose compositions for delivery of pharmaceutical agents such as bisantrene or a derivative or analog thereof comprising a microcapsule, wherein the microcapsule includes a hardening agent that is a volatile silicone fluid. U.S. Pat. No. 5,070,082 to Murdock et al., U.S. Pat. No. 5,077,282 to Murdock et al., and U.S. Pat. No. 5,077,283 to Murdock et al. disclose prodrug forms of poorly soluble hydrophobic drugs, including bisantrene and derivatives and analogs, that are salts of a phosphoramidic acid. U.S. Pat. No. 5,116,827 to Murdock et al. and U.S. Pat. No. 5,212,291 to Murdock et al. disclose prodrug forms of poorly soluble hydrophobic drugs, including bisantrene and derivatives and analogs, that are quinolinecarboxylic acid derivatives. U.S. Pat. No. 5,378,456 to Tsou includes compositions containing an anthracene antitumor agent, such as bisantrene or a derivative or analog thereof, in which the bisantrene or derivative or analog thereof is conjugated to or admixed with a divinyl ether-maleic acid (MVE) copolymer. U.S. Pat. No. 5,609,867 to Tsou discloses polymeric 1,4-bis derivatives of bisantrene and copolymers of bisantrene and another monomer, such as a dianhydride.

Methods and compositions described herein can use a derivative or analog of bisantrene in place of bisantrene itself. Derivatives and analogs of bisantrene are described in in U.S. Pat. Nos. 10,500,19 and 10,548,876, by Garner et al.

Methods for Treating ccRCC and Other VHL(−) Cancers

Surprisingly, the present studies have shown that protein kinase inhibitors and bisantrene (or pharmaceutically acceptable salts thereof) act synergistically against ccRCC cell liones. It is contemplated that derivatives of bisantrene possessing substantially the same activity will also act synergistically with protein kinases in methods and compositions according to the present invention.

Thus, one aspect of the invention provides a method of treating a patient with ccRCC or other VHL(−) cancer, especially protein kinase inhibitor resistant ccRCC, said method comprising administering to said patient a therapeutically effective amount of a protein kinase inhibitor, such as a tyrosine kinase inhibitor, and a therapeutically effective amount of at least a second agent comprising bisantrene or a derivative thereof, or a pharmaceutically acceptable salt of bisantrene or derivative thereof.

The protein kinase inhibitor may inhibit one or more potential protein kinase targets associated with ccRCC and other VHL(−) cancers. According to certain embodiments, the protein kinase inhibitor inhibits. According to certain embodiments, the protein kinase inhibitor inhibits at least one or more of VEGFR1, VEGFR2, VEGFR3, DDR1, DDR2, RET and RIPK2. According to certain embodiments, the protein kinase inhibitor inhibits at least one or more of VEGFR1, VEGFR2, VEGFR3.

According to certain embodiments the at least one protein kinase inhibitor is selected from the group consisting of: pazopanib; lenvatinib; cabozantinib; everolimus; sorafenib; sunitinib; temsirolimus; mitomycin C; axitinib; tivozanib; and belzutifan.

According to specific embodiments, the at least one protein kinase inhibitor is selected from the group consisting of: pazopanib; lenvatinib; cabozantinib; sorafenib; sunitinib; mitomycin C; axitinib; tivozanib; and belzutifan.

According to more specific embodiments of the present invention, the at least one protein kinase inhibitor is selected from the group consisting of: pazopanib; lenvatinib; cabozantinib; sorafenib; sunitinib; axitinib; and tivozanib.

According to even more specific embodiments of the present invention, the at least one protein kinase inhibitor is selected from the group consisting of: pazopanib; lenvatinib; and cabozantinib.

According to alternative embodiments, the at least one protein kinase inhibitor is an inhibitor of mTOR, optionally selected from temsirolimus and everolimus.

According to specific embodiments of the present invention, the second agent is bisantrene or a pharmaceutically acceptable salt thereof.

According to certain embodiments, the bisantrene can be administered as a drug compound or as a component of a pharmaceutical composition, as discussed further below.

According to certain specific embodiments, the method comprises administering to said patient a therapeutically effective amount of one protein kinase inhibitor and a therapeutically effective amount of bisantrene or a pharmaceutically acceptable salt thereof.

The pharmaceutical compositions and medicaments of the present invention may be administered to a subject by standard enteral or parenteral routes, including, but not limited to, injection (such as intravenous, subcutaneous, intramuscular, bolus, etc.), or by, for example, topical, oral, sublingual, nasal, pulmonary, otic, rectal or vaginal administration routes. In some embodiments, pharmaceutical compositions according to the invention may be administered to a subject by themselves or in combination with other pharmaceutical composition(s). In the latter case, the administration may be simultaneous or sequential, or administration of the pharmaceutical composition(s) may be independent of one another.

In certain embodiments bisantrene (or derivative, or pharmaceutically acceptable salt of either) is administered intravenously, either centrally or peripherally, including by intramuscular, subcutaneous and/or intradermal injection.

In general, the pharmaceutical compositions and medicaments of the present invention can be administered in a manner compatible with the route of administration and physical characteristics of the subject (including health status) and in such a way that the desired effect(s) are induced (i.e. therapeutically effective and/or preventative). For example, the appropriate dosage may depend on a variety of factors including, but not limited to, a subject's physical characteristics (e.g. age, weight, sex), whether the composition or medicament is being used as single agent, the progression (i.e. pathological state) of the disease or disorder being treated, and other factors readily apparent to those of ordinary skill in the art.

Suitable dosages, dosage frequencies, dosage durations, and routes of administration for chemotherapeutic agents are known in the art. As suggested by FIG. 16, bisantrene, derivatives of bisantrene, or pharmaceutically acceptable salts of either can be administered in the same pharmaceutical composition as a protein kinase inhibitor, in a separate composition as, but simultaneously with the protein kinase inhibitor, or at a different time. If the bisantrene, derivative of bisantrene, or pharmaceutically acceptable salt of bisantrene or derivative of bisantrene, is administered at a different time than the protein kinase inhibitor, it can either be administered before or after the protein kinase inhibitor and/or at different timings, being administered according to different timing and/or frequency regimes. One of ordinary skill in the art can determine a suitable schedule for administration based on variables such as the age, weight, and sex of the subject, the susceptibility of the subject to side effects of the active agents, genetic markers, the dose of active agent(s), the subjects history with prior active agent(s), and other pharmacokinetic parameters such as heart, liver or kidney function.

The methods and compositions provided herein enable a subject to receive a therapy more frequently without having the dosage regimen significantly altered by the risk of side-effects, such as cardiotoxicity. The dose(s) of a protein kinase inhibitor and bisantrene, derivative of bisantrene, or pharmaceutically acceptable salt of bisantrene or derivative of bisantrene, may be administered to a subject in one or more doses per day. In some cases, the daily dose of the chemotherapeutic agent may be administered together with bisantrene, derivative of bisantrene, or pharmaceutically acceptable salt of either, in a single dose.

The pharmaceutical compositions described herein may be administered to a patient one or more times per day. In some cases, the pharmaceutical composition may be administered to a patient once per day. In some cases, the pharmaceutical composition may be administered to a patient at least 2 times, 3 times, 4 times 5 times, or 6 times per day. For example, a pharmaceutical composition may be administered to a patient 3 times per day.

In methods described herein, suitable dosages of bisantrene (or a derivative of bisantrene, or a pharmaceutically acceptable salt of either) can be determined by one of ordinary skill in the art. The selected dosage level depends upon a variety of pharmacokinetic factors including the amount of active agent(s) being administered, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000, and Gilman et al., (Eds), (1990), “Goodman And Gilman's: The Pharmacological Bases of Therapeutics”, Pergamon Press. Optimal dosages for a given set of conditions can be ascertained by those skilled in the art using conventional dosage-determination tests in view of the experimental data for an agent.

According to certain embodiments, administration of bisantrene is performed at a dosage of from about 0.1 mg/m²/day to about 400 mg/m²/day, such as from about 0.2 mg/m²/day to about 300 mg/m²/day, from about 0.5 mg/m²/day to about 200 mg/m²/day, from about 0.5 mg/m²/day to about 100 mg/m²/day, from about 0.5 mg/m²/day to about 50 mg/m²/day, from about 0.5 mg/m²/day to about 30 mg/m²/day, from about 0.5 mg/m²/day to about 20 mg/m²/day, from about 1.0 mg/m²/day to about 10 mg/m²/day, from about 1.0 mg/m²/day to about 8 mg/m²/day, about 1 mg/m²/day, about 2 mg/m²/day, about 3 mg/m²/day, about 4 mg/m²/day, about 5 mg/m²/day, about 6 mg/m²/day, about 7 mg/m²/day, or about 10 mg/m²/day. In some embodiments, bisantrene is administered daily or weekly, once every two weeks, once every three weeks, once every four weeks, over a period of, for example, 1 day, 2 days, 3 days, 4 days, 5 days, 6 days, 7 days, 8 days, 9 days, 10 days, 11 days, 12 days, 13 days, 14 days, 21 days, 28 days, 35 days, 42 days, 49 days, 56 days or 63 days. In certain embodiments, bisantrene is administered once or multiple times over a period of 28 days, optionally once or multiple times daily or weekly, once every two weeks, once every three weeks, once every four weeks, at a dosage of about 20-50 mg/m²/28 days. Administration of pharmaceutically acceptable salts of bisantrene or derivatives or pharmaceutically acceptable salts thereof may be performed at similar dosage rates, adjusted for molar equivalence.

Dosing regimens for protein kinase inhibitors will depend on the particular protein kinase inhibitor, and have been published for known protein kinase inhibitors, including for treatment of ccRCC and other VHL(−) cancers. In addition, suitable dosages of a given protein kinase inhibitor can be readily determined by one of ordinary skill in the art using known approaches and techniques as discussed above in relation to bisantrene.

According to certain embodiments, and as a result of the synergistic biologic activity observed when bisantrene (or derivative of bisantrene, or a pharmaceutically acceptable salt of either) is administered with a protein kinase inhibitor, a therapeutic outcome may be achieved more effectively for a given dose rate for that protein kinase inhibitor. This biologic activity of the bisantrene (or derivative of bisantrene, or a pharmaceutically acceptable salt of either) may also allow for use, or expanded use of protein kinase inhibitors which at normal dosages may have deleterious side-effects, such as cardiotoxicity.

Alternatively, when administered in combination with bisantrene (or derivative of bisantrene, or a pharmaceutically acceptable salt of either), the protein kinase inhibitor may be administered to a patient at a lower dosage than it would normally be administered, over a longer period while maintaining a comparable ongoing therapeutic outcome. This is particularly important for active agents with deleterious side-effects, such as cardiotoxicity. Thus, in certain embodiments of methods according to the present invention, when administered in combination with bisantrene (or derivative of bisantrene, or a pharmaceutically acceptable salt of either), the protein kinase inhibitor may be administered at a rate which is significantly lower than the recommended dosage rate for that protein kinase inhibitor when administered alone, such as at a rate which is at least 10%, 20%, 30%, 40%, 50%, 60% or 70% lower than the dosage rate for that protein kinase inhibitor when administered alone.

### Additional Active Agents

Programmed cell death 1 ligand 1 (PDL1) and its receptor programmed cell death 1 (PD1) regulate the activation of immune cells. Checkpoint inhibitors can assist in suppressing this regulation, allowing immune cells to destroy tumours. An emerging strategy is the combination of checkpoint inhibitors with protein kinase inhibitors, especially protein kinase inhibitors that target VEGFR and other kinases involved in angiogenesis, with the underlying rationale that angiogenesis is important in establishing an immunosuppressive environment. Known checkpoint inhibitors include antibodies that target PD1 or PDL1.

The FDA has approved two PD-1 inhibitors (nivolumab and pembrolizumab), one PD-L1 inhibitor (avelumab), and one CTLA-4 inhibitor (ipilimumab) for use in ccRCC. Other PD-1/PD-L1 immune checkpoint inhibitors of potential consideration include atezolizumab, bevacizumab, cemiplimab, dostarlimab, and durvalumab.

In addition, some studies have shown that protein kinase inhibitors can themselves stimulate immune responses against tumours, which may in turn result in synergistic responses when combined with checkpoint inhibitors.

Similarly, immunomodulators such as cytokines that regulate immune cell maturation, growth and activation, including interleukin-2 (Aldesleukin/Proleukin®), interferon alpha-2a, interferon alpha-2b (and Peginterferon alpha-2b), and Granulocyte-macrophage colony-stimulating factor (GM-CSF) have also been approved for treatment of various cancers via immunostimulation. Interleukin-2 (or the recombinant human version Aldesleukin/Proleukin®) has been approved for the treatment of RCC.

Thus, methods according to the present invention may further comprise administration of at least one additional therapeutic agent for the treatment of ccRCC or other VHL(−) cancer.

According to certain embodiments the additional therapeutic agent is a checkpoint inhibitor or an immunomodulator such as those discussed above. According to certain embodiments the at least one additional therapeutic agent comprises a checkpoint inhibitor selected from the group comprising nivolumab, pembrolizumab, avelumab, ipilimumab, atezolizumab, bevacizumab, cemiplimab, dostarlimab, and durvalumab or an immunomodulator selected from interleukin-2 (Aldesleukin/Proleukin®), interferon alpha-2a, interferon alpha-2b (and Peginterferon alpha-2b), and GM-CSF. According to certain embodiments, the at least one additional therapeutic agent comprises a checkpoint inhibitor selected from the group comprising nivolumab, pembrolizumab, avelumab, and ipilimumab. According to certain embodiments the at least one additional therapeutic agent comprises interleukin-2 (Aldesleukin/Proleukin®).

Suitable dosages, dosage frequencies, dosage durations, and routes of administration for these additional agents are known in the art. As suggested by FIG. 17, these additional agents can either be administered simultaneously with the bisantrene or the derivative or analog of bisantrene, or at a different time than the bisantrene or the derivative or analog of bisantrene. If the additional agent is administered at a different time than the bisantrene or the derivative or analog of bisantrene, it can either be administered before or after the bisantrene or the derivative or analog of bisantrene and/or at different timings, being administered according to different timing and/or frequency regimes. Similarly, and independently of bisantrene considerations, these additional agents can either be administered simultaneously with the protein kinase inhibitor, or at a different time than the protein kinase inhibitor. If the additional agent is administered at a different time than the protein kinase inhibitor, it can either be administered before or after the protein kinase inhibitor and/or at different timings, being administered according to different timing and/or frequency regimes. One of ordinary skill in the art can determine a suitable schedule for administration based on variables such as the age, weight, and sex of the patient, the severity of the cancer, genetic markers such as further described below, and pharmacokinetic parameters such as liver and kidney function.

FIG. 16 shows a prophetic example of a bisantrene and targeted agent combination trial design to overcome therapy resistance in ccRCC and other VHL(−) cancers.

FIG. 17 shows a prophetic example of a bisantrene and checkpoint inhibitor combination trial design to overcome immunotherapy resistance in ccRCC and other VHL(−) cancers.

Pharmaceutical Compositions for the Treatment of ccRCC and Other VHL(−) Cancers

The present invention also provides pharmaceutical compositions for the treatment of ccRCC or other VHL(−) cancer comprising at least one protein kinase inhibitor, as described above, and a second agent comprising bisantrene or a derivative thereof, or a pharmaceutically acceptable salt of bisantrene or derivative thereof. As described above, the compositions of the invention may comprise an additional agent selected from checkpoint inhibitors and immunomodulators.

According to certain embodiments the protein kinase inhibitor is a tyrosine kinase inhibitor. According to certain embodiments, the protein kinase inhibitor inhibits at least one or more of VEGFR1, VEGFR2, VEGFR3, DDR1, DDR2, RET and RIPK2. According to certain embodiments, the protein kinase inhibitor inhibits at least one or more of VEGFR1, VEGFR2, VEGFR3.

According to certain embodiments the at least one protein kinase inhibitor is selected from the group consisting of: pazopanib; lenvatinib; cabozantinib; everolimus; sorafenib; sunitinib; temsirolimus; mitomycin C; axitinib; tivozanib; and belzutifan.

According to specific embodiments of the present invention, the at least one protein kinase inhibitor is selected from the group consisting of: pazopanib; lenvatinib; and cabozantinib.

According to specific embodiments of the present invention, the second agent is bisantrene or a pharmaceutically acceptable salt thereof.

According to specific embodiments of the present invention, the composition may comprise a therapeutically effective amount of one protein kinase inhibitor and a therapeutically effective amount of bisantrene or a pharmaceutically acceptable salt thereof.

Compositions according to the present invention may further comprise administration of at least one additional therapeutic agent for the treatment of ccRCC or other VHL(−) cancer.

According to certain embodiments the additional therapeutic agent is a checkpoint inhibitor or an immunomodulator such as those discussed above. According to certain embodiments the at least one additional therapeutic agent comprises a checkpoint inhibitor selected from the group comprising nivolumab, pembrolizumab, avelumab, ipilimumab, atezolizumab, bevacizumab, cemiplimab, dostarlimab, and durvalumab or an immunomodulator selected from interleukin-2 (Aldesleukin/Proleukin®)), interferon alpha-2a, interferon alpha-2b (and Peginterferon alpha-2b), and GM-CSF. According to certain embodiments, the at least one additional therapeutic agent comprises a checkpoint inhibitor selected from the group comprising nivolumab, pembrolizumab, avelumab, and ipilimumab. According to certain embodiments the at least one additional therapeutic agent comprises interleukin-2 (Aldesleukin/Proleukin®).

Compositions according to the present invention may be administered by any route, and in a form suitable for that route, as known in the art. Thus, compositions according to the present invention may be adapted for administration by enteral or parenteral routes, including by injection (such as intravenous, subcutaneous, intramuscular, bolus, etc.), or by, for example, topical, oral, sublingual, nasal, pulmonary, otic, rectal or vaginal administration routes.

Typically, the pharmaceutical compositions described herein include at least one pharmaceutically acceptable carrier or excipient and/or diluents. For preparing the pharmaceutical compositions and medicaments, inert, pharmaceutically acceptable carriers can be either solid or liquid. Liquid form preparations include solutions, suspensions and emulsions, for example water or water-propylene glycol solutions for parenteral injection. Also included are solid form preparations, such as tablets, or amorphous or crystalline powders, including lyophilized preparations, that are intended to be converted, shortly before use, to liquid form preparations for either oral or injection administration. Such liquid forms include solutions, suspensions and emulsions. Examples of pharmaceutically acceptable carriers and methods of manufacture for various compositions may be found in, for example, “Remington: The Science and Practice of Pharmacy”, Mack Publishing Co., 20^{th} ed., 2000, and Gilman et al., (Eds), (1990), “Goodman And Gilman's: The Pharmacological Bases of Therapeutics”, Pergamon Press.

Pharmaceutically acceptable carriers and excipients include:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) an oil;
(xvi) a penetration enhancer;
(xvii) a polymer;
(xviii) a stiffening agent;
(xix) a protein;
(xx) a carbohydrate;
(xxi) a bulking agent; and
(xxii) a lubricating agent.

Other pharmaceutically acceptable carriers and excipients known in the art may be used.

The carriers, diluents, excipients and adjuvants must be “acceptable” in terms of being compatible with the other ingredients of the composition or medicament, and are generally not deleterious to the subject thereof. Non-limiting examples of pharmaceutically acceptable carriers or diluents are demineralised or distilled water; saline solution; vegetable-based oils such as peanut oil, safflower oil, olive oil, cottonseed oil, maize oil; sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxylpropyl-methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from about 10% to about 99.9% by weight of the composition, vaccine or medicament.

For administration as an injectable solution or suspension, non-toxic parenterally acceptable diluents or carriers can include, Ringer's solution, isotonic saline, phosphate buffered saline, ethanol and 1,2 propylene glycol. Methods for preparing parenterally administrable pharmaceutical compositions and medicaments are apparent to those of ordinary skill in the art, and are described in more detail in, for example, “Remington: The Science and Practice of Pharmacy”, Mack Publishing Co., 20^{th} ed., 2000, and Gilman et al., (Eds), (1990), “Goodman And Gilman's: The Pharmacological Bases of Therapeutics”, Pergamon Press.

For oral administration, some examples of suitable carriers, diluents, excipients and adjuvants include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl stearate which delay disintegration.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

In certain embodiments, the pharmaceutical composition may comprise a liposome. A liposomal formulation suitable for bisantrene or a cardioprotective derivative thereof comprises small unilamellar or multilamellar liposomes of size range between 0.01 and 100 μM, and between about 50-95% liposome-entrapped bisantrene, composed of hydrogenated soy phosphatidylcholine, distearoyl phosphatidylglycerol, and cholesterol of natural or synthetic origin lipids, in aqueous solution which can be reconstituted from a lyophilized form to an injectable liposome suspension. The composition is prepared by reconstituting a lyophilized bisantrene/liposome composition to a liposome concentrate, then diluting the concentrate for parenteral administration for the treatment of melanoma.

In yet another embodiment, the pharmaceutical composition may comprise a complex with a beta-cyclodextrin. A liposomal formulation suitable for bisantrene, a therapeutically active derivative of bisantrene, or a pharmaceutically acceptable salt of bisantrene or derivative thereof, comprises a complex formed in aqueous solution which may be reconstituted from a lyophilized form to an injectable suspension. One such composition is prepared by reconstituting a lyophilized bisantrene/beta-cyclodextrin composition to a concentrate, then diluting the concentrate for parenteral administration. Beta-cyclodextrin complexes and methods for preparing such complexes are known in the art and are described in., for example, WO 2019/073296 by Rothman.

Various formulations suitable for use in the administration of bisantrene, a therapeutically active derivative of bisantrene, or a pharmaceutically acceptable salt of bisantrene or derivative thereof, are known in the art. U.S. Pat. No. 4,784,845 to Desai et al. discloses a composition for delivery of a hydrophobic drug (i.e., bisantrene or a cardioprotective derivative thereof) comprising: (i) the hydrophobic drug; (ii) an oleaginous vehicle or oil phase that is substantially free of butylated hydroxyanisole (BHA) or butylated hydroxytoluene (BHT); (iii) a co-surfactant or emulsifier; (iv) a co-surfactant or auxiliary emulsifier; and (v) benzyl alcohol as a co-solvent. U.S. Pat. No. 4,816,247 by Desai et al. discloses a composition for delivery by intravenous, intramuscular, or intraarticular routes of hydrophobic drugs (such as bisantrene or a cardioprotective derivative or analog thereof) comprising: (i) the hydrophobic drug; (ii) a pharmaceutically acceptable oleaginous vehicle or oil selected from the group consisting of: (a) naturally occurring vegetable oils and (b) semisynthetic mono-, di-, and triglycerides, wherein the oleaginous vehicle or oil is free of BHT or BHA; (iii) a surfactant or emulsifier; (iv) a co-surfactant or emulsifier; (v) an ion-pair former selected from C₆-C₂₀ saturated or unsaturated aliphatic acids when the hydrophobic drug is basic and a pharmaceutically acceptable aromatic amine when the hydrophobic drug is acidic; and (vi) water.

According to certain embodiments, compositions according to the present invention may be adapted for administration of bisantrene at a dosage of from about 0.1 mg/m²/day to about 100 mg/m²/day, such as from about 0.2 mg/m²/day to about 50 mg/m²/day, from about 0.5 mg/m²/day to about 20 mg/m²/day, from about 1.0 mg/m²/day to about 10 mg/m²/day, from about 1.0 mg/m²/day to about 8 mg/m²/day, about 1 mg/m²/day, about 2 mg/m²/day, about 3 mg/m²/day, about 4 mg/m²/day, about 5 mg/m²/day, about 6 mg/m²/day, about 7 mg/m²/day, or about 10 mg/m²/day. In some embodiments, compositions according to the present invention may be adapted for administration of bisantrene once or multiple times daily or weekly, once every two weeks, once every three weeks, once every four weeks, over a period of, for example, 1 day, 2 days, 3 days, 4 days, 5 days, 6 days, 7 days, 8 days, 9 days, 10 days, 11 days, 12 days, 13 days, 14 days, 21 days, 28 days, 35 days, 42 days, 49 days, 56 days or 63 days. In certain embodiments, compositions according to the present invention may be adapted for administration of bisantrene over a period of 28 days, optionally once or multiple times daily or weekly, once every two weeks, once every three weeks, once every four weeks, at a dosage of about 20-50 mg/m²/28 days. Compositions according to the present invention comprising pharmaceutically acceptable salts of bisantrene or therapeutically active derivatives or pharmaceutically acceptable salts thereof may be adapted to deliver similar dosage rates, adjusted for molar equivalence.

Dosing regimens for protein kinase inhibitors will depend on the particular protein kinase inhibitor, and have been published for known protein kinase inhibitors, including for treatment of ccRCC. In addition, suitable dosages of a given protein kinase inhibitor can be readily determined by one of ordinary skill in the art using known approaches and techniques as discussed above in relation to bisantrene. Compositions according to the invention may be formulated to include the protein kinase(s) at their recommended dosage rates, or different dosage rates as discussed below.

According to certain embodiments, and as a result of the synergistic biologic activity observed when bisantrene (or derivative of bisantrene, or a pharmaceutically acceptable salt of either) is administered with a protein kinase inhibitor, the dosage of protein kinase in a composition according to the present invention may be lower than it would be when administered to a patient by itself. This is particularly important for active agents with deleterious side-effects, such as cardiotoxicity. Thus, in certain embodiments of compositions according to the present invention, the protein kinase inhibitor may be included in the composition at a dosage which is significantly lower than the recommended dosage rate for that protein kinase inhibitor when administered alone, such as at a rate which is at least 10%, 20%, 30%, 40%, 50%, 60% or 70% lower than the dosage rate for that protein kinase inhibitor when administered alone.

Similarly, where compositions according to the present invention include an additional therapeutic agent, being a checkpoint inhibitor and/or an immunomodulator, that additional therapeutic agent may be included in the composition at its known recommended rate(s) for the treatment of ccRCC and other VHL(−) cancers, or even lower rates if synergy amongst the components is determined.

Uses of Compositions for the Manufacture of Medicaments for the Treatment of ccRCC and Other VHL(−) Cancers

The present invention also provides uses of compositions according to the invention for the manufacture of a medicament for the treatment of ccRCC or other VHL(−) cancer, especially protein kinase resistant ccRCC in a patient.

The parameters for such manufacture, including protein kinase inhibitors, bisantrene, bisantrene derivatives and salts thereof, as well as checkpoint inhibitors and immunomodulators as optional additional therapeutic agent(s), and dosages/dosing for all actives, dosage forms and other components are described above.

Kits for the Treatment of ccRCC and Other VHL(−) Cancers

The present invention also provides kits for the treatment of ccRCC or other VHL(−) cancer, especially protein kinase resistant ccRCC, the kit comprising at least one protein kinase inhibitor and at least a second agent comprising bisantrene or a derivative thereof, or a pharmaceutically acceptable salt of bisantrene or derivative thereof.

The parameters for such kits, including protein kinase inhibitors, bisantrene, bisantrene derivatives and salts thereof, as well as checkpoint inhibitors and immunomodulators as optional additional therapeutic agent(s), and dosages/dosing for all actives, dosage forms and other components are described above.

In some cases, the kit may also comprise vials, tubes, needles, packaging, or other materials.

Kits with unit doses of one or more of the active agents described herein, usually in injectable doses, are provided. Such kits may include containers containing unit doses, informational package inserts describing the use and attendant benefits of the drugs in treating the disease, and optionally appliance(s) or device(s) for delivery of the composition.

The kit may further comprise any device suitable for administration of the composition. For example, a kit may comprise a needle suitable for intravenous administration.

In some cases, kits may be provided with instructions. The instructions may be provided in the kit or they may be accessed electronically. The instructions may provide information on how to use the compositions of the present disclosure. The instructions may further provide information on how to use the devices of the present disclosure. The instructions may provide information on how to perform the methods of the disclosure. In some cases, the instructions may provide dosing information. The instructions may provide drug information such as the mechanism of action, the formulation of the drug, adverse risks, contraindications, and the like. In some cases, the kit is purchased by a physician or health care provider for administration at a clinic or hospital. In some cases, the kit is purchased by a laboratory and used for screening candidate compounds.

Preferred forms of the present invention will now be described, by way of example only, with reference to the following examples, including comparative data, and which are not to be taken to be limiting to the scope or spirit of the invention in any way.

### EXAMPLES

### Example 1: Cell Viability Assays and Analysis

Cell viability was determined using a resazurin metabolic activity assay. Cells were seeded in duplicate wells of 96-well microtitre plates at 1×10³ cells/well (786-O, RCC4 EV, RCC4 VHL), or 3×10³ cells/well (Caki-1, Caki-2) and cultured for 24 h.

Zantrene (bisantrene dihydrochloride) was reconstituted in dimethylsulphoxide (DMSO) at 20 mM or in a 5% Captisol solution at 15 mM. Everolimus (RAD001), sunitinib, sorafenib, pazopanib, lenvantinib (mesylate), cabozantinib (BMS-907351) were reconstituted in DMSO at 17.2 mM (lenvantinib) or 100 mM (all others).

Drugs were then diluted in media and added to wells, and cells cultured for a further 72 h.

Human renal cell lines were cultured in a humidified chamber at 37° C. with 5% CO₂ in the medias listed in Table 1 below:

TABLE 1
Cell culture media and supplements for renal cell lines.

| Cell Line(s) | Base Media | Supplements |
|---|---|---|
| 786-O | RPMI-1640 (with | 10% foetal bovine serum (FBS), 20 mM |
| | GlutaMAX) | HEPES, 1 mM sodium pyruvate |
| Caki-1, | McCoy's 5A | 10% FBS, 2 mM L-glutamine |
| Caki-2 | | |
| RCC4 EV, | DMEM (high | 10% FBS, 2 mM L-glutamine, 20 mM |
| RCC4 VHL | glucose) | HEPES |

Viability was determined using the fluorogenic viability dye resazurin (Ex 544 nm, Em 590 nm; 0.6 mM resazurin, 78 μM methylene blue, 1 mM potassium hexacyanoferrate (III), 1 mM potassium hexacyanoferrate (II) trihydrate (Sigma Aldrich), dissolved in PBS [3]). Resazurin is metabolised into the red-fluorescent resorufin by metabolically active cells. Fluorescence was measured 5 h post-addition of resazurin solution (1:10, v/v) at 544 nm excitation/590 nm emission on a FLUOstar OPTIMA plate reader (BMG LabTechnologies). Graphpad Prism 9 software was used to generate graphs. Drug IC₅₀ values were determined by cubic spline-lowess regression analysis using Prism 9. At least three independent replicates were performed for each cell line and each drug combination, and data is represented as mean±standard error of the mean (SEM). The culture conditions for each cell line are described below.

Clonogenicity assays—Clonogenicity assays were used to determine the colony-forming ability of cells treated with Zantrene. RCC4EV and RCC4 VHL Cells were seeded into 6-well plates at 1000 cells/well and allowed to adhere for 24 h. Bisantrene was diluted in media and added to wells, and cells were cultured for 96 h. Drug-containing media was removed and replaced with freshmedia (without drug) and cultured for an additional 96 h to allow the formation of cell colonies. At endpoint, media was removed from wells and cells washed with cold PBS twice. Cells were fixed with ice-cold methanol for 10 minutes on ice followed immediately by staining with crystal violet solution (0.5% crystal violet, 25% methanol in PBS) at room temperature. Excess crystal violet solution was washed away and pictures of plates capturedon a ChemiDoc MP Imaging System (Bio-Rad). Images were analysed using the ColonyArea plugin [Guzman C. et al (2014), “ColonyArea: an ImageJ plugin to automatically quantify colony formation in clonogenic assays”, PLoS One, 9(3):e92444] for ImageJ [Schneider C. A. et at (2012), “NIH Image to ImageJ: 25 years of image analysis”, Nat Methods 9(7):671-675] and the percent area of the well filled by colonies determined and presented as % of untreated cells using Prism 9. Four independent replicates were performed for each cell line and data is represented as mean±SEM.

Synergy Analysis—for combination drug treatments, three different synergy analyses have been conducted, including the fraction product method of Webb (Webb J (1963), “Effect of more than one inhibitor” In: Hochster E. R., Quastel J. (eds). “Enzymes and metabolic inhibitors”, Academic Press: New York), the Chou-Talalay (combination index; CI)(Chou T-C. (2020), “Theoretical Basis, Experimental Design, and Computerized Simulation of Synergism and Antagonism in Drug Combination Studies”, Pharmacological Reviews, 58(3):621-681), and the BLISS synergy method (Bliss (1939) “Cl: The toxicity of poisons applied jointly” Ann App Biol, 26:585-615) using SynergyFinder 2.0 software (Ianevski A, Giri A K, Aittokallio T (2020): “SynergyFinder 2.0: visual analytics of multi-drug combination synergies”, Nucleic Acids Res., 48(W1):W488-W493). Detail on each of the methods is provided below.

Fractional Product Method of Webb: Webb, in 1963 (Webb J (1963), “Effect of more than one inhibitor” In: Hochster E. R., Quastel J. (eds). “Enzymes and metabolic inhibitors”, Academic Press: New York), introduced a method that was later termed the “fractional product” method (Chou T-C. (2020), “Theoretical Basis, Experimental Design, and Computerized Simulation of Synergism and Antagonism in Drug Combination Studies”, Pharmacological Reviews, 58(3):621-681). It estimates the expected additive effect of two drugs using the fractional product of the effect of each drug alone, ie:

$$1 - Fa ( Drug 1 + Drug 2 ) = ( 1 - FaDrug 1 ) * ( 1 - FaDrug 2 )$$

Where Fa=the fraction of cells affected, expressed as a decimal.

An observed Fa(Drug1+Drug2) value greater than the expected Fa(Drug1+Drug2) value result indicates synergy, whereas an observed Fa(Drug1+Drug2) lower than the expected value indicates antagonism. The method of Webb therefore takes into account the potency of each drug at a particular dose, however does not consider the shape of the dose-response curve as a whole [10]. The result is considered as:
Result values <−0.1 indicates synergy
Result values between −0.1 and 0.1 indicate additivity
Result values >0.1 indicates antagonism

Chou-Talalay Method: In 1984, Chou and Talalay put forward what is the most widely used synergy evaluation method to date (Chou TC and Talalay P (1984), “Quantitative analysis of dose-effect relationships: the combined effects of multiple drugs or enzyme inhibitors. Advances in enzyme regulation”, 1984, 22:27-55). Using the Henderson-Hasselbalch, Michaelis-Menten, Scatchard, and Hill equations, Chou and Talaly derived the “median-effect” equation to model the median effect dose and sigmoidicity of the dose-response curve, using the inputted doses and corresponding dose-effects. Chou and Talalay also coined the term “CombinationIndex (CI)” to evaluate the degree of synergy between two compounds, defined as:

$$CI = \frac{1}{{DRI}_{DrugA}} + \frac{1}{{DRI}_{DrugB}}$$

wherein:
DRI=the Drug Reduction Index, or the fold reduction in dose of Drug A effected by the presence of Drug B;
CI of less than 0.9 indicates synergy;
CI between 0.9 to 1.1 indicates an additive interaction; and
CI greater than 1.1 indicates antagonism.

The Cl at any Fa can be estimated using the median effect equation.

Both the Chou Talalay and Webb methods are widely used and robust synergy determination methods. While the Chou Talalay method is considered a more sophisticated model, it is recommended to use a constant dose ratio between the two drugs in combination. The method of Webb is therefore recommended when this assumption is not met.

Bliss method: SynergyFinder (version 2.0) is a stand-alone web-application for interactive analysis and visualization of multi-drug (2 or more drugs) combination profiling data (Ianevski A, Giri A K, Aittokallio T (2020): “SynergyFinder 2.0: visual analytics of multi-drug combination synergies”, Nucleic Acids Res., 48(W1):W488-W493). The degree of combination synergy, or antagonism, is quantified by comparing the observed drug combination response against the expected response, calculated using a reference model that assumes no interaction between drugs. Within SynergyFinder the Bliss model was used. This model quantifies the degree of synergy as the multiplicative effect of single drugs as if they acted independently. This independence model assumes a stochastic process in which two drugs elicit their effects independently, and the expected combination effect can be calculated based on the probability of independent events. When the synergy score is:
less than −10: the interaction between two drugs is likely to be antagonistic;
from −10 to 10: the interaction between two drugs is likely to be additive; and
greater than 10: the interaction between two drugs is likely to be synergistic.

### Example 2: Combination of Zantrene with Currently Used Protein Kinase Inhibitors

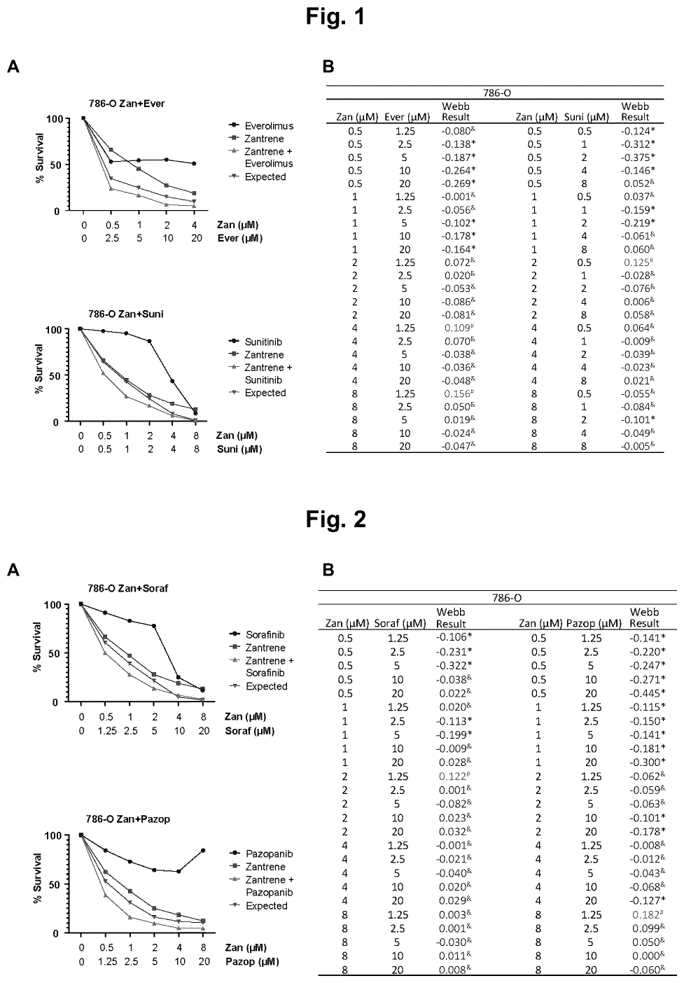

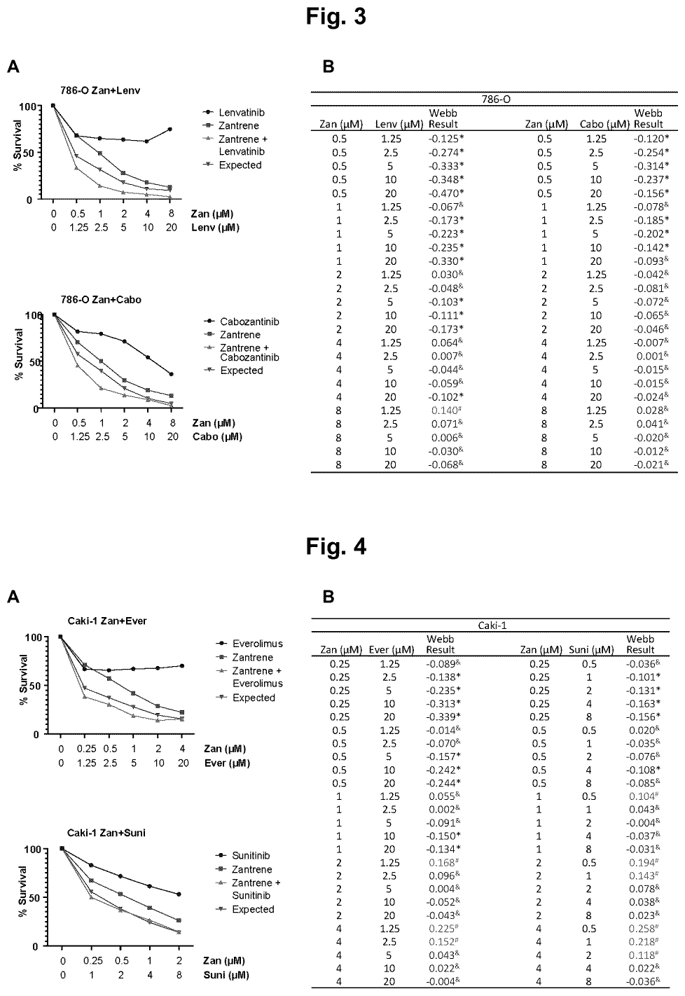

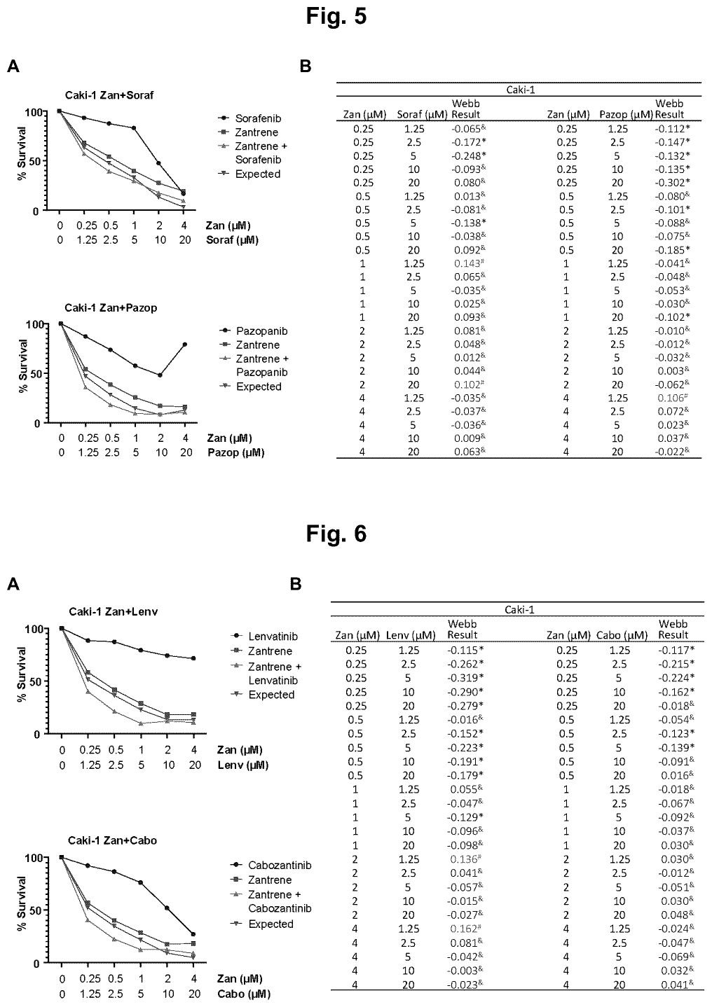

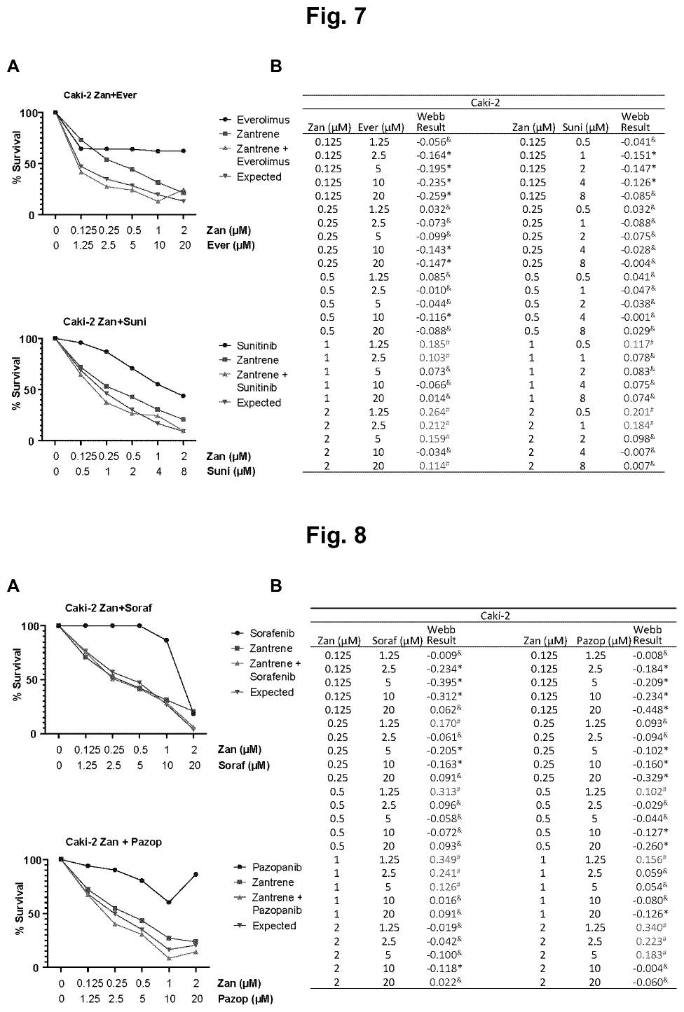

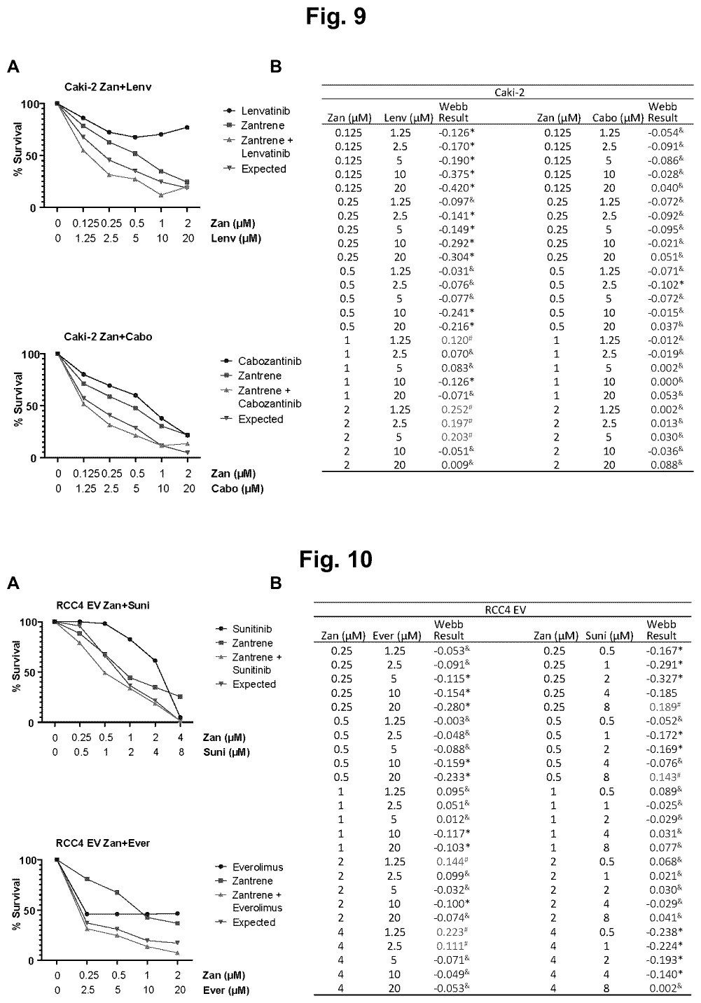

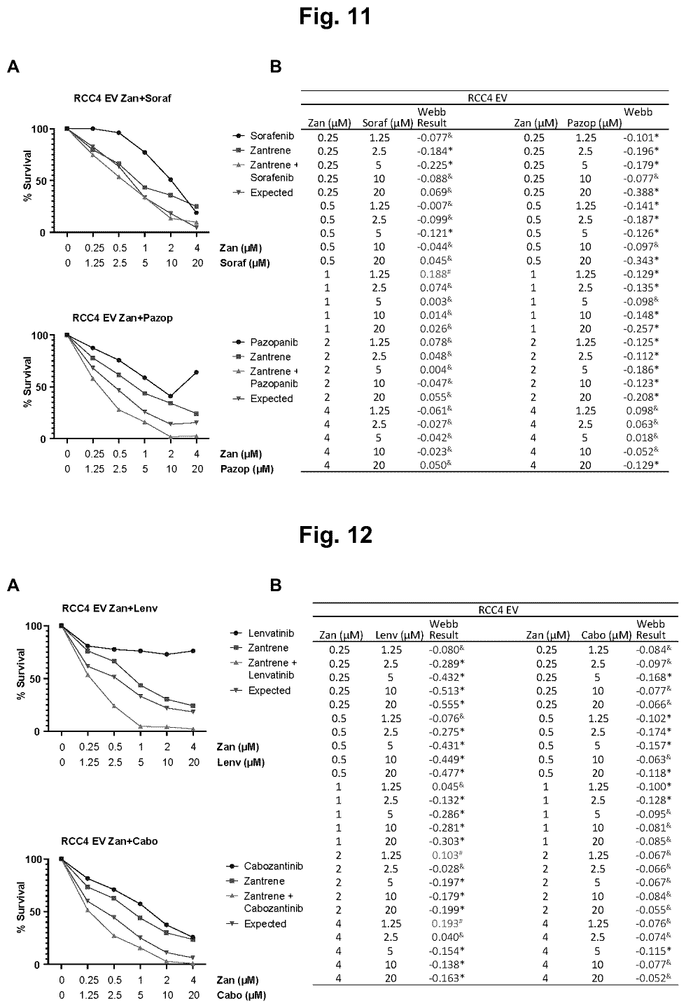

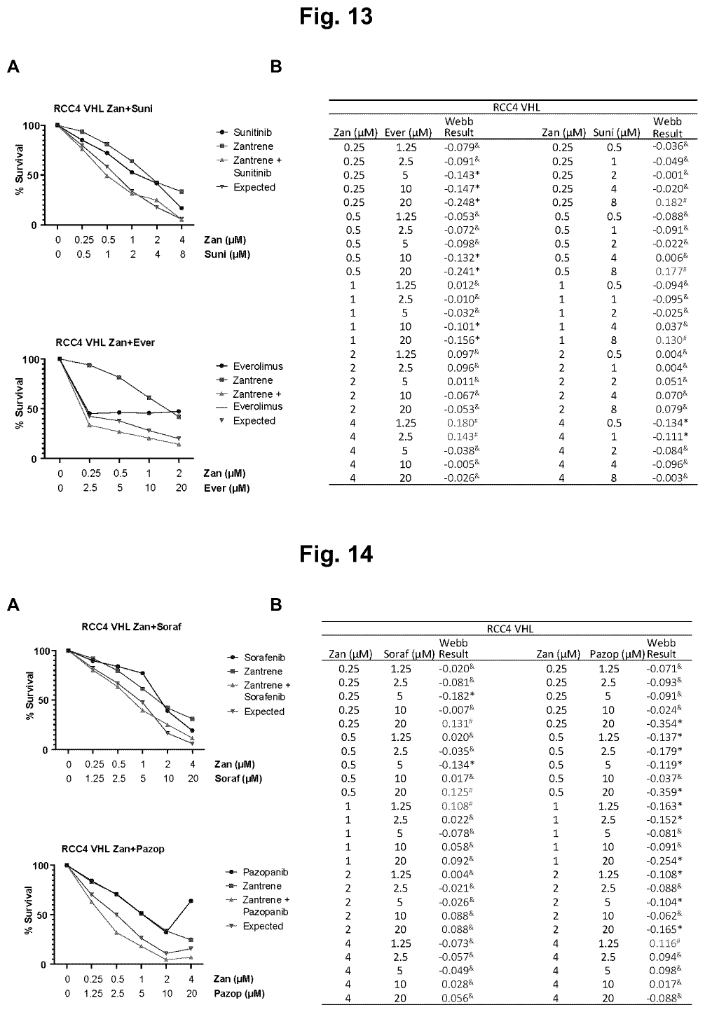

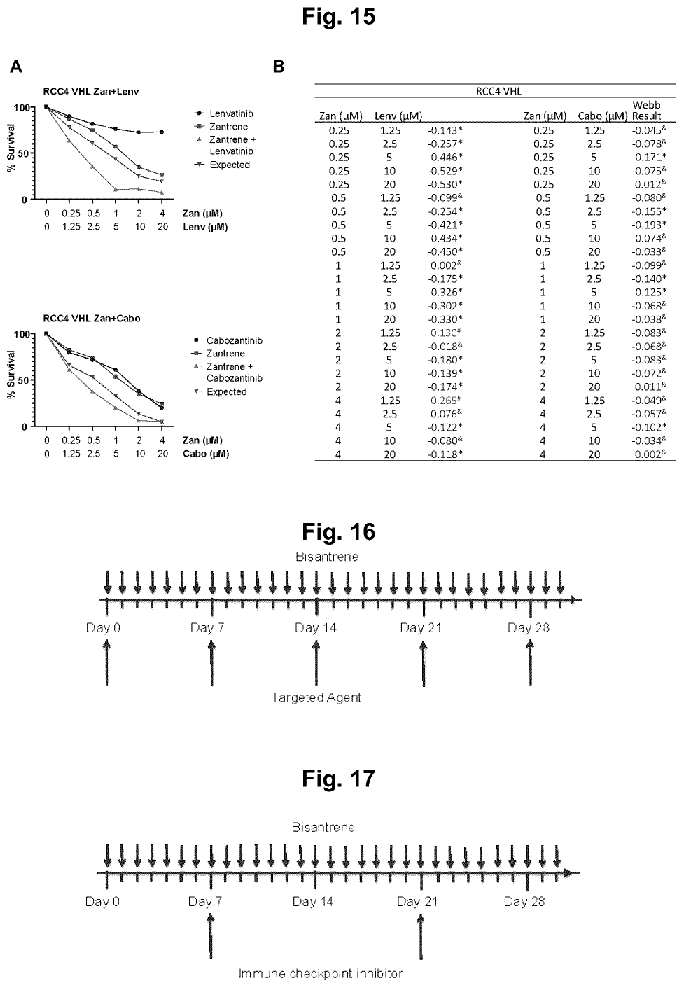

To determine the effects of the combined drug treatments, three different methods were utilised. The method of Webb analysis revealed synergy across multiple drug doses for zantrene and protein kinase inhibitors in the 786-0 cell line (FIG. 1-3). The strongest synergy was observed for zantrene in combination with lenvatinib, cabozantinib and pazopanib. Synergy was also observed with doses of sunitunib, sorafenib, and everolimus, most often at lower doses. Similar results were observed across the other 4 cell lines, with lenvatinib and cabozantinib followed by pazopanib, consistently displaying the strongest synergy with zantrene (FIG. 4-15).

Analysis using the Bliss method similarly revealed overall synergistic effects of zantrene with lenvatinib and pazopanib in all 5 cell lines (Table 2). This method further revealed everolimus to be synergistic in all 5 cell lines, and cabozantinib in all lines (although this was marginal for Caki-2 cells). Mild synergy was observed for sunitinib and sorafenib (Table 2). The Bliss analysis can also provide a score for the most synergistic area across the drug doses, and this suggests areas of synergy for the drugs tested across all cell lines (Table 3). Individual Bliss scores for each dose combination further revealed multiple doses that were synergistic across most cell lines.

TABLE 2
Overall synergy score of bisantrene-drug combinations
as determined by Bliss synergy analysis

| Drug combination | 786-0 | Caki-1 | Caki-2 | RCC4-EV | RCC4-VHL |
|---|---|---|---|---|---|
| Everolimus + | 18.15* | 19.88* | 15.45* | 17.58* | 17.74* |
| Zantrene | | | | | |
| Sunitinib + | 12.26* | 6.42^{&} | 5.26^{&} | 8.01^{&} | 5.21^{&} |
| Zantrene | | | | | |
| Sorafenib + | 10.40* | 7.86^{&} | 5.07^{&} | 5.09^{&} | 4.41^{&} |
| Zantrene | | | | | |
| Pazopanib + | 21.86* | 16.24* | 13.95* | 22.34* | 17.84* |
| Zantrene | | | | | |
| Lenvatinib + | 24.49* | 20.68* | 17.93* | 36.42* | 29.32* |
| Zantrene | | | | | |
| Cabozantinib + | 18.07* | 15.08* | 8.64^{&} | 19.15* | 15.21* |
| Zantrene | | | | | |

Values >10 are considered synergistic (*); values between −10 to 10 are additive (^{&}); values below −10 are considered antagonistic (^{#}).

TABLE 3
Most synergistic area score of bisantrene-drug combinations
as determined by Bliss synergy analysis

| Drug combination | 786-0 | Caki-1 | Caki-2 | RCC4-EV | RCC4-VHL |
|---|---|---|---|---|---|
| Everolimus + | 27.12* | 28.81* | 23.00* | 22.31* | 27.52* |
| Zantrene | | | | | |
| Sunitinib + | 24.37* | 14.24* | 15.73* | 14.79* | 12.97* |
| Zantrene | | | | | |
| Sorafenib + | 23.69* | 18.66* | 11.54* | 10.41* | 12.14* |
| Zantrene | | | | | |
| Pazopanib + | 30.44* | 22.50* | 16.90* | 26.02* | 22.96* |
| Zantrene | | | | | |
| Lenvatinib + | 35.02* | 30.28* | 21.24* | 44.15* | 36.05* |
| Zantrene | | | | | |
| Cabozantinib + | 30.13* | 26.66* | 18.54* | 26.86* | 24.50* |
| Zantrene | | | | | |

Values >10 are considered synergistic (*); values between −10 to 10 are additive (^{&}); values below −10 are considered antagonistic (^{#}).

Finally, Chou-Talalay analyses was used to quantify the combination effects of zantrene and each anti-RCC drug. This analysis is less stringent than the method of Webb or the method of Bliss analysis and identified synergy at most doses for almost all combinations. When considering the synergy score at the IC₅₀ for both drugs, synergy was predicted for zantrene with sunitinib, pazopanib, lenvatinib and cabozantinib (Table 4).

TABLE 4
Combination index of bisantrene-drug combinations
determined by Chou Talalay analysis

| | ED₅₀ | | | | |
|---|---|---|---|---|---|
| Drug Combination | 786-O | Caki-1 | Caki-2 | RCC-EV | RCC-VHL |
| Everolimus − | 0.35* | NA | 0.27* | 6.28E+11^{#} | 6.17E+57^{#} |
| Zantrene | | | | | |
| Sunitinib − | 0.76* | 0.61* | 0.67* | 0.69* | 0.69* |
| Zantrene | | | | | |
| Sorafenib − | 0.75* | 0.69* | 0.92^{&} | 0.83* | 0.9* |
| Zantrene | | | | | |
| Pazopanib − | 0.51* | 0.71* | 0.56* | 0.42* | 0.44* |
| Zantrene | | | | | |
| Lenvatinib − | 0.37* | 0.61* | 0.28* | 0.3* | 0.28* |
| Zantrene | | | | | |
| Cabozantinib − | 0.53* | 0.5* | 0.56* | 0.52* | 0.58* |
| Zantrene | | | | | |

Combination index was calculated from the ED50 using Chou-Talalay analysis; antagonism(^{#}); additive (^{&}); moderate synergism (*).

Lenvatinib, cabozantinib and pazopanib displayed the strongest synergy with zantrene in the ccRCC cells tested. This may indicate a common cellular target for these drugs that is synergistically lethal with zantrene. These drugs are all VEGFR inhibitors that inhibit angiogenesis. In addition to inhibiting VEGFR on endothelial cells, these multi-kinase inhibitors also inhibit tumour growth due to tumour-cell intrinsic inhibition of other kinases. Given our assays only consist of tumour cells, the synergistic cytoxicity observed with zantrene must be due to kinase inhibition within the tumour cells. Common targets for lenvatinib, cabozantinib and pazopanib include DDR2, DDR1, RET and RIPK2. RET was also identified as a target of sunitinib. The Caki-1 cell line was further added to the 4-cell line pool for analysis of some drugs, including cabozantinib, and this further revealed AXL and MET as cabozantinib targets.

### Example 3: Zantrene Kills Clear Cell Renal Cell Carcinoma Cells and is More Toxic to VHL(−) Cells

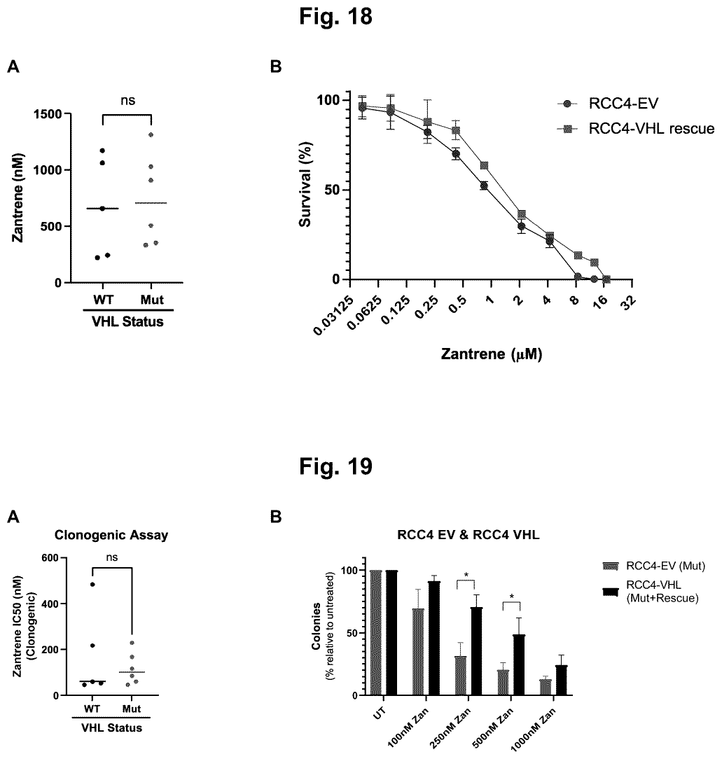

The results shown in Example 2 indicated greater synergy between bisantrene and protein kinase inhibitors when mutant VHL(−) cells (RCC4 EV cells) were treated with that combination compared to a VHL rescue line (RCC4 VHL). Those results are consistent with earlier observations where bisantrene was shown to not only be toxic to all kidney cancer cell lines (data not shown), but to be surprisingly more toxic to RCC4 EV cells, which are VHL(−) mutant cells, as compared to the isogenic rescue (VHL(+)) RCC4 VHL cells, as shown in Table 5 and illustrated in FIG. 18.

TABLE 5
Cytotoxic IC₅₀ values of bisantrene in renal
cancer RCC4 EV (VHL(−); mutant/Mut) and
VHL rescue RCC4 VHL (Wildtype/WT) cell lines

| Cell Line | Renal Cell Type | VHL status | bisantrene IC₅₀ (nM) |
|---|---|---|---|
| RCC4 EV | ccRCC | Mut | 907 |
| RCC4 VHL | ccRCC | Mut + WT | 1170 |

Greater lethality between VHL loss and bisantrene was also observed for RCC4 EV cells, as compared to RCC4 VHL cells using long term clonogenic cell growth assays (cell colony formation) as described in Example 1, which better measures a drug's effect on cancer cell growth rather than cell killing.

The RCC-4 EV (VHL mutant) cells were significantly more sensitive (2.9×) to bisantrene than the wild-type VHL rescue cells (Table 6 & FIG. 19).

TABLE 6
Clonogenic IC₅₀ of Zantrene in renal RCC4 EV (VHL(−);
mutant/Mut) and VHL rescue RCC4 VHL (Wildtype/WT) cell lines

| Cell Line | Renal Cell Type | VHL status | bisantrene IC50 (nM) |
|---|---|---|---|
| RCC4 EV | ccRCC | Mut | 167 |
| RCC4 VHL | ccRCC | Mut + WT | 483 |

### Advantages of the Invention

The present invention provides a new paradigm for treating of ccRCC and other VHL(−) cancers by the administration of bisantrene, a antineoplastic agent that has multiple mechanisms of action, including DNA intercalation, inhibition of topoisomerase, inhibition of the fat and obesity associated protein (FTO), and activation of the immune system. Bisantrene is well tolerated and, in particular, lacks the cardiotoxicity that is characteristic of some other anthracene derivatives. Bisantrene can be used together with other therapeutic agents that are used for the treatment of ccRCC and other VHL(−) cancers.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. As used herein the use of the term “comprising” as a transitional phrase in claims is intended to include therein the use of the transitional phrases “consisting essentially of” or “consisting of” if the narrower transitional phrases are not expressly excluded. Although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

1. A method of treating a patient with ccRCC or other VHL(−) cancer, said method comprising administering to said patient a therapeutically effective amount of a protein kinase inhibitor and a therapeutically effective amount of at least a second agent comprising bisantrene or a derivative thereof, or a pharmaceutically acceptable salt of bisantrene or derivative thereof.

2. The method of claim 1, wherein said protein kinase inhibitor is a tyrosine kinase inhibitor.

3. The method of claim 1 or claim 2, wherein said protein kinase inhibitor inhibits one or more of VEGFR1, VEGFR2, VEGFR3, DDR1, DDR2, RET and RIPK2.

4. The method of claim 1, wherein the at least one protein kinase inhibitor is selected from the group consisting of:
(a) pazopanib;
(b) lenvatinib;
(c) cabozantinib;
(d) everolimus;
(e) sorafenib;
(f) sunitinib;
(g) temsirolimus;
(h) mitomycin C;
(i) axitinib;
(j) tivozanib; and
(k) belzutifan.

5. The method of claim 1, wherein said at least one protein kinase inhibitor is selected from the group consisting of:
(a) pazopanib;
(b) lenvatinib; and
(c) cabozantinib.

6. The method of any one of claims 1 to 5, wherein said second agent is bisantrene or a pharmaceutically acceptable salt thereof.

7. The method of any one of claims 1 to 6 which comprises administering to said patient a therapeutically effective amount of one protein kinase inhibitor and a therapeutically effective amount of bisantrene or a pharmaceutically acceptable salt thereof.

8. The method of any one of claims 1 to 7 which comprises treatment of ccRCC, optionally protein kinase inhibitor resistant ccRCC.

9. The method of any one of claims 1 to 8, further comprising administration of at least one additional therapeutic agent for the treatment of ccRCC or other VHL(−) cancer.

10. The method of claim 9, wherein the additional therapeutic agent is a checkpoint inhibitor drug or an immunomodulator.

11. The method of claim 10, wherein the at least one additional therapeutic agent is selected from the group consisting of:
(a) atezolizumab;
(b) avelumab;
(c) bevacizumab;
(d) cemiplimab;
(e) dostarlimab;
(f) durvalumab;
(g) interleukin-2;
(h) ipilimumab;
(i) nivolumab;
(j) pembrolizumab; and
(k) proleukin.

12. The method of claim 1 comprising administering said at least one protein kinase inhibitor to said patient prior to, simultaneously with, or after administration of said second agent to said patient.

13. The method of any one of claims 1 to 10, wherein said at least one protein kinase inhibitor and the second agent are administered to said patient at the same time, optionally in a single composition.

14. The method of any one of claims 1 to 13, wherein said treatment has synergistic results against ccRCC and other VHL(−) cancers compared to a method wherein said at least one protein kinase or said second agent is administered alone.

15. The method of any one of claims 1 to 14, wherein the dose of said at least one protein kinase inhibitor is at least 25% lower than the dose required of said at least one protein kinase inhibitor agent when administered without said second agent to achieve the same targeted outcome.

16. A pharmaceutical composition for the treatment of ccRCC or other VHL(−) cancer comprising at least one protein kinase inhibitor and a second agent comprising bisantrene or a derivative thereof, or a pharmaceutically acceptable salt of bisantrene or derivative thereof.

17. The composition of claim 16, wherein said protein kinase inhibitor is a tyrosine kinase inhibitor.

18. The composition of claim 17, wherein said protein kinase inhibitor inhibits one or more of VEGFR1, VEGFR2, VEGFR3, DDR1, DDR2, RET and RIPK2.

19. The composition of claim 16, wherein said at least one protein kinase inhibitor is selected from the group consisting of:
(a) pazopanib;
(b) lenvatinib;
(c) cabozantinib;
(d) everolimus;
(e) sorafenib;
(f) sunitinib;
(g) temsirolimus;
(h) mitomycin C;
(i) axitinib;
(j) tivozanib; and
(k) belzutifan.

20. The composition of claim 16, wherein said at least one protein kinase inhibitor is selected from the group consisting of:
(a) pazopanib;
(b) lenvatinib; and
(c) cabozantinib.

21. The composition of any one of claims 16 to 20, further comprising at least one additional therapeutic agent for the treatment of ccRCC and other VHL(−) cancers.

22. The composition of claim 21, wherein the additional therapeutic agent is a checkpoint inhibitor drug or an immunomodulator.

23. The composition of claim 22, wherein the at least one additional therapeutic agent is selected from the group consisting of:
(c) atezolizumab;
(d) avelumab;
(c) bevacizumab;
(d) cemiplimab;
(e) dostarlimab;
(f) durvalumab;
(g) interleukin-2;
(h) ipilimumab;
(i) nivolumab;
(j) pembrolizumab; and
(k) proleukin.

24. The composition of any one of claims 16 to 23, wherein said second agent is bisantrene or a pharmaceutically acceptable salt thereof.

25. The composition of any one of claims 16 to 24 which comprises a therapeutically effective amount of one protein kinase inhibitor and a therapeutically effective amount of bisantrene or a pharmaceutically acceptable salt thereof.

26. The composition of any one of claims 16 to 25 for the treatment of ccRCC, optionally protein kinase inhibitor resistant ccRCC.

27. The composition of any one of claim 16 having synergistic activity against ccRCC or other VHL(−) cancer compared to said at least one protein kinase or said second agent when administered alone.

28. Use of a composition according to any one of claims 16 to 26 for the manufacture of a medicament for the treatment of ccRCC or other VHL(−) cancer in a patient.

29. The use of claim 28, wherein said medicament is for the treatment of ccRCC, optionally protein kinase inhibitor resistant ccRCC.

30. The use of claim 28 or claim 29, wherein said medicament comprises one protein kinase inhibitor and bisantrene or a pharmaceutically acceptable salt thereof.

31. A kit for the treatment of ccRCC or other VHL(−) cancer, said kit comprising at least one protein kinase inhibitor and at least a second agent comprising bisantrene or a derivative thereof, or a pharmaceutically acceptable salt of bisantrene or derivative thereof.

32. The kit of claim 31, wherein said protein kinase inhibitor is a tyrosine kinase inhibitor.

33. The kit of claim 31 or claim 32, wherein said protein kinase inhibitor inhibits one or more of VEGFR1, VEGFR2, VEGFR3, DDR1, DDR2, RET and RIPK2.

34. The kit of claim 31 wherein the at least one additional therapeutic agent is selected from the group consisting of:
(a) pazopanib;
(b) lenvatinib;
(c) cabozantinib;
(d) everolimus;
(e) sorafenib;
(f) sunitinib;
(g) temsirolimus;
(h) mitomycin C;
(i) axitinib;
(j) tivozanib; and
(k) belzutifan.

35. The kit of claim 31, wherein said at least one protein kinase inhibitor is selected from the group consisting of:
(a) pazopanib;
(b) lenvatinib; and
(c) cabozantinib.

36. The kit of any one of claims 31 to 35, wherein said second agent is bisantrene or a pharmaceutically acceptable salt thereof.

37. The kit of any one of claims 31 to 36 which comprises one protein kinase inhibitor and bisantrene or a pharmaceutically acceptable salt thereof.

38. The kit of any one of claims 31 to 37, further comprising at least one additional therapeutic agent for the treatment of ccRCC or other VHL(−) cancer.

39. The kit of claim 38, wherein the additional therapeutic agent is a checkpoint inhibitor drug or an immunomodulator.

40. The kit of claim 39, wherein the at least one additional therapeutic agent is selected from the group consisting of:
(a) atezolizumab;
(b) avelumab;
(c) bevacizumab;
(d) cemiplimab;
(e) dostarlimab;
(f) durvalumab;
(g) interleukin-2;
(h) ipilimumab;
(i) nivolumab;
(j) pembrolizumab; and
(k) proleukin.

41. The kit of any one of claims 31 to 40 for the treatment of ccRCC, optionally protein kinase inhibitor resistant ccRCC.

44. The kit of any one of claims 31 to 41 comprising instructions for administering said at least one protein kinase inhibitor to said patient prior to, simultaneously with, or after administering said second agent to said patient.

43. The kit of any one of claims 31 to 42, comprising instructions for administering said at least one protein kinase inhibitor and the second agent to said patient at the same time.
